(19) United States
(12) Patent Application Publication
(10) Pub. No.: US 2007/0179618 A1
(43) Pub. Date: Aug. 2, 2007

(54) Intervertebral prosthetic disc

(75) Inventors: Hai H. Trieu, Cordova, TN (US); Eric S. Heinz, Memphis, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/343,935

(22) Filed: Jan. 31, 2006

(51) Int. Cl. A61F 2/44

(52) U.S. Cl. 623/17.14

(57) ABSTRACT

An intervertebral prosthetic disc is disclosed and can be installed within an intervertebral space between a first vertebra and a second vertebra. The intervertebral prosthetic disc can include a first component that can have a first compliant layer that can be configured to engage the first vertebra and at least partially conform to a shape of the first vertebra. Further, the intervertebral prosthetic disc can include a second component that is configured to engage the second vertebra.

### FIELD OF THE DISCLOSURE

The present disclosure relates generally to orthopedics and spinal surgery. More specifically, the present disclosure relates to intervertebral prosthetic discs.

### BACKGROUND

In human anatomy, the spine is a generally flexible column that can take tensile and compressive loads. The spine also allows bending motion and provides a place of attachment for keels, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral-bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively.

### BRIEF DESCRIPTION OF THE DRAWINGS

FIG. 1 is a lateral view of a portion of a vertebral column;

FIG. 2 is a lateral view of a pair of adjacent vertebrae;

FIG. 3 is a top plan view of a vertebra;

FIG. 4 is an anterior view of a first embodiment of an intervertebral prosthetic disc;

FIG. 5 is an exploded anterior view of the first embodiment of the intervertebral prosthetic disc;

FIG. 6 is a lateral view of the first embodiment of the intervertebral prosthetic disc;

FIG. 7 is an exploded lateral view of the first embodiment of the intervertebral prosthetic disc;

FIG. 8 is a plan view of a superior half of the first embodiment of the intervertebral prosthetic disc;

FIG. 9 is another plan view of the superior half of the first embodiment of the intervertebral prosthetic disc;

FIG. 10 is a plan view of an inferior half of the first embodiment of the intervertebral prosthetic disc;

FIG. 11 is a plan view of an inferior half of the first embodiment of the intervertebral prosthetic disc;

FIG. 12 is an exploded lateral view of the first embodiment of the intervertebral prosthetic disc installed within an intervertebral space between a pair of adjacent vertebrae;

FIG. 13 is an anterior view of the first embodiment of the intervertebral prosthetic disc installed within an intervertebral space between a pair of adjacent vertebrae;

FIG. 14 is an anterior view of a second embodiment of an intervertebral prosthetic disc;

FIG. 15 is an exploded anterior view of the second embodiment of the intervertebral prosthetic disc;

FIG. 16 is a lateral view of the second embodiment of the intervertebral prosthetic disc;

FIG. 17 is an exploded lateral view of the second embodiment of the intervertebral prosthetic disc;

FIG. 18 is a plan view of a superior half of the second embodiment of the intervertebral prosthetic disc;

FIG. 19 is another plan view of the superior half of the second embodiment of the intervertebral prosthetic disc;

FIG. 20 is a plan view of an inferior half of the second embodiment of the intervertebral prosthetic disc;

FIG. 21 is another plan view of the inferior half of the second embodiment of the intervertebral prosthetic disc;

FIG. 22 is an anterior view of a third embodiment of an intervertebral prosthetic disc;

FIG. 23 is an exploded anterior view of the third embodiment of the intervertebral prosthetic disc;

FIG. 24 is a lateral view of the third embodiment of the intervertebral prosthetic disc;

FIG. 25 is an exploded lateral view of the third embodiment of the intervertebral prosthetic disc;

FIG. 26 is a plan view of a superior half of the third embodiment of the intervertebral prosthetic disc;

FIG. 27 is another plan view of the superior half of the third embodiment of the intervertebral prosthetic disc;

FIG. 28 is a plan view of an inferior half of the third embodiment of the intervertebral prosthetic disc;

FIG. 29 is another plan view of the inferior half of the third embodiment of the intervertebral prosthetic disc;

FIG. 30 is a lateral view of a fourth embodiment of an intervertebral prosthetic disc;

FIG. 31 is an exploded lateral view of the fourth embodiment of the intervertebral prosthetic disc;

FIG. 32 is a anterior view of the fourth embodiment of the intervertebral prosthetic disc;

FIG. 33 is a perspective view of a superior component of the fourth embodiment of the intervertebral prosthetic disc;

FIG. 34 is a perspective view of an inferior component of the fourth embodiment of the intervertebral prosthetic disc;

FIG. 35 is a lateral view of a fifth embodiment of an intervertebral prosthetic disc;

FIG. 36 is an exploded lateral view of the fifth embodiment of the intervertebral prosthetic disc;

FIG. 37 is a anterior view of the fifth embodiment of the intervertebral prosthetic disc;

FIG. 38 is a perspective view of a superior component of the fifth embodiment of the intervertebral prosthetic disc; and

FIG. 39 is a perspective view of an inferior component of the fifth embodiment of the intervertebral prosthetic disc.

### DETAILED DESCRIPTION OF THE DRAWINGS

An intervertebral prosthetic disc is disclosed and can be installed within an intervertebral space between a first vertebra and a second vertebra. The intervertebral prosthetic disc can include a first component that can have a first compliant layer that can be configured to engage the first vertebra and at least partially conform to a shape of the first vertebra. Further, the intervertebral prosthetic disc can include a second component that is configured to engage the second vertebra.

In another embodiment, an intervertebral prosthetic disc is disclosed and can be installed within an intervertebral space between an inferior vertebra and a superior vertebra. The intervertebral prosthetic disc can include an inferior support plate that can have an inferior bearing surface. Moreover, an inferior compliant layer can be disposed on the inferior bearing surface. Also, an inferior embedded layer can be disposed within the inferior bearing surface. The intervertebral prosthetic disc can also include a superior support plate that can have a superior bearing surface. A superior compliant layer can be disposed on the superior bearing surface. Further, a superior embedded layer can be disposed within the superior bearing surface.

In yet another embodiment, an intervertebral prosthetic disc is disclosed and can be installed within an intervertebral space between an inferior vertebra and a superior vertebra. The intervertebral prosthetic disc can include a superior component and the superior component can include a superior support plate that can have a superior bearing surface. Additionally, a superior compliant layer can be disposed on the superior bearing surface. The intervertebral disc can also include an inferior component that can have an inferior support plate and the inferior support plate can have an inferior bearing surface. An inferior compliant layer can be disposed on the inferior bearing surface. Moreover, a nucleus can be disposed between the superior component and the inferior component. The nucleus can be configured to allow relative motion between the superior component and the inferior component.

Description of Relevant Anatomy

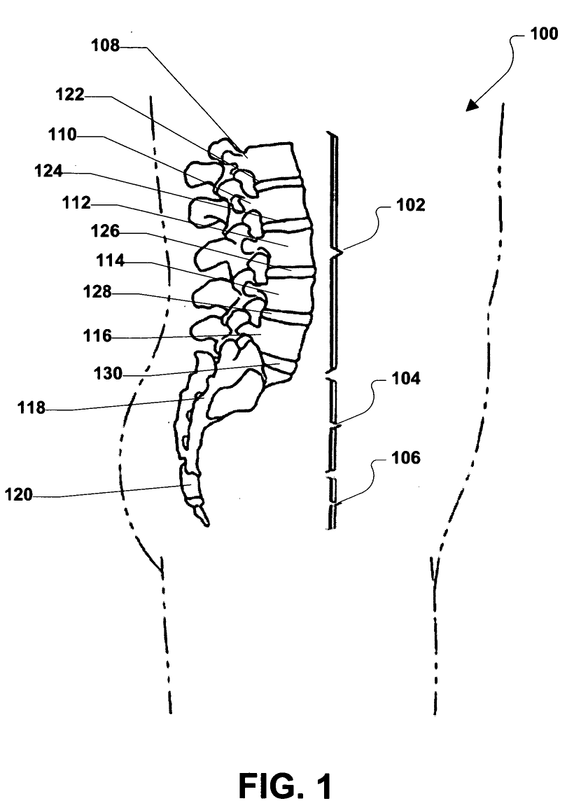

Referring initially to FIG. 1, a portion of a vertebral column, designated 100, is shown. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of replacement, that intervertebral lumbar disc 122, 124, 126, 128, 130 can be at least partially removed and replaced with an intervertebral prosthetic disc according to one or more of the embodiments described herein. In a particular embodiment, a portion of the intervertebral lumbar disc 122, 124, 126, 128, 130 can be removed via a discectomy, or a similar surgical procedure, well known in the art. Further, removal of intervertebral lumbar disc material can result in the formation of an intervertebral space (not shown) between two adjacent lumbar vertebrae.

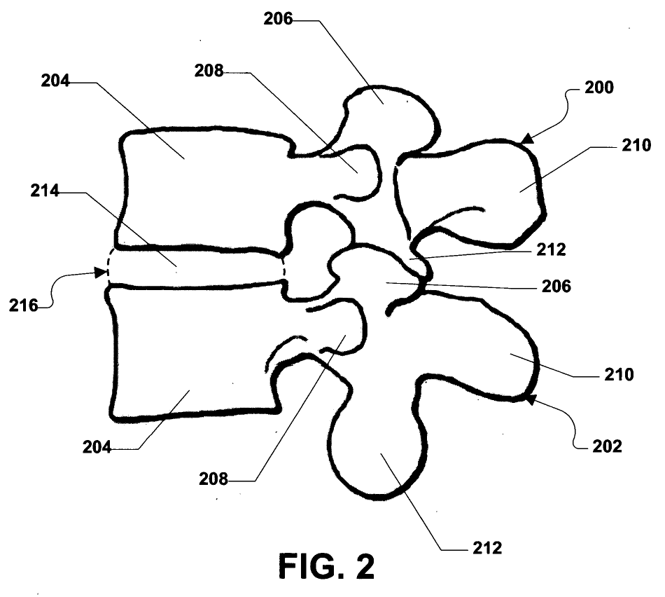

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines). As described in greater detail below, an intervertebral prosthetic disc according to one or more of the embodiments described herein can be installed within the intervertebral space 212 between the superior vertebra 200 and the inferior vertebra 202.

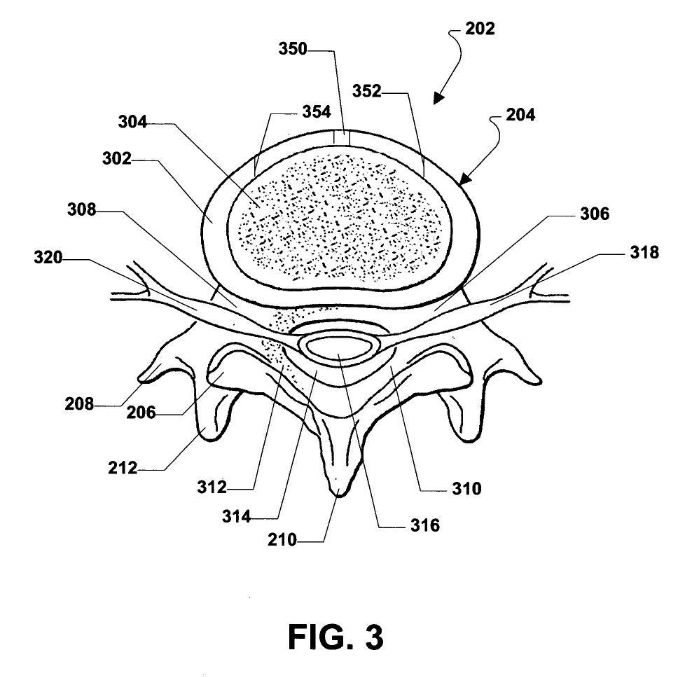

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

FIG. 3 further depicts a keel groove 350 that can be established within the cortical rim 302 of the inferior vertebra 202. Further, a first corner cut 352 and a second corner cut 354 can be established within the cortical rim 302 of the inferior vertebra 202. In a particular embodiment, the keel groove 350 and the corner cuts 352, 354 can be established during surgery to install an intervertebral prosthetic disc according to one or more of the embodiments described herein. The keel groove 350 can be established using a keel cutting device, e.g., a keel chisel designed to cut a groove in a vertebra, prior to the installation of the intervertebral prosthetic disc. Further, the keel groove 350 is sized and shaped to receive and engage a keel, described in detail below, that extends from an intervertebral prosthetic disc according to one or more of the embodiments described herein. The keel groove 350 can cooperate with a keel to facilitate proper alignment of an intervertebral prosthetic disc within an intervertebral space between an inferior vertebra and a superior vertebra.

Description of a First Embodiment of an Intervertebral Prosthetic Disc

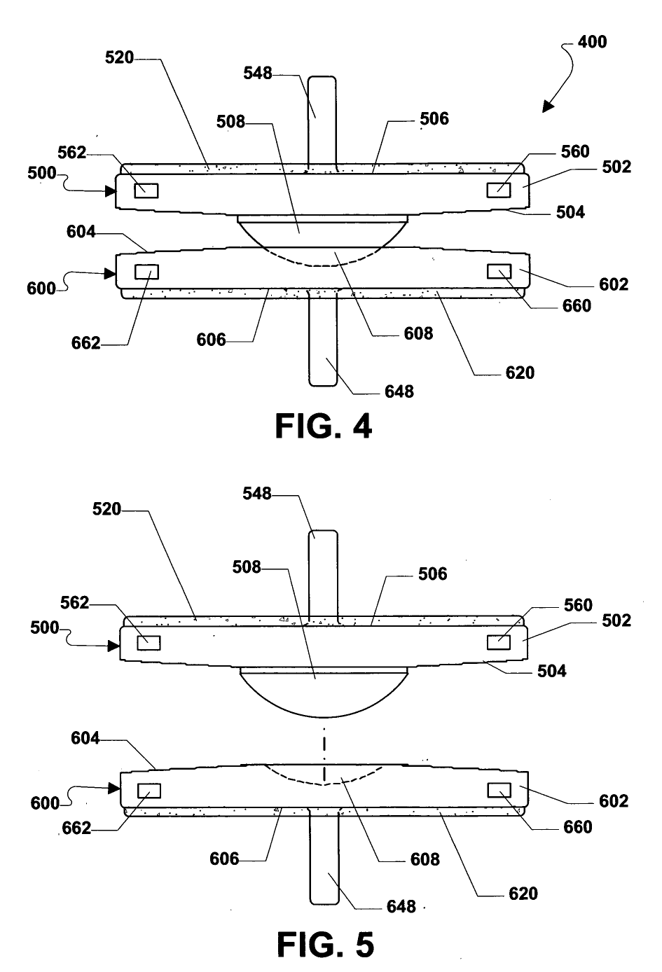

Referring to FIGS. 4 through 11 a first embodiment of an intervertebral prosthetic disc is shown and is generally designated 400. As illustrated, the intervertebral prosthetic disc 400 includes a superior component 500 and an inferior component 600. In a particular embodiment, the components 500, 600 can be made from one or more extended use biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 500, 600 can be made from any other substantially rigid biocompatible materials.

In a particular embodiment, the superior component 500 includes a superior support plate 502 that has a superior articular surface 504 and a superior bearing surface 506. In a particular embodiment, the superior articular surface 504 can be generally curved and the superior bearing surface 506 can be substantially flat. In an alternative embodiment, the superior articular surface 504 can be substantially flat and at least a portion of the superior bearing surface 506 can be generally curved.

As illustrated in FIG. 4 through FIG. 7, a projection 508 extends from the superior articular surface 504 of the superior support plate 502. In a particular embodiment, the projection 508 has a hemi-spherical shape. Alternatively, the projection 508 can have an elliptical shape, a cylindrical shape, or other arcuate shape. Moreover, the projection 508 can be formed with a groove 510.

As further illustrated, the superior component 500 includes a superior compliant layer 520 that can be affixed to, attached to, or otherwise deposited on, the superior bearing surface 506. The superior compliant layer 520 can be chemically bonded to the superior bearing surface 506, e.g., using an adhesive or another chemical bonding agent. Further, the superior compliant layer 520 can be mechanically anchored to the superior bearing surface 506, e.g., using hook-and-loop fasteners, or another type of fastener.

Before the superior compliant layer 520 is deposited, or otherwise affixed to the superior bearing surface 506, the superior bearing surface 506 can be modified to promote adhesion of the superior compliant layer 520 to the superior bearing surface 506. For example, the superior bearing surface 506 can be roughened to promote adhesion of the superior compliant layer 520. For example, the roughening process can include acid etching; knurling; application of a bead coating, e.g., cobalt chrome beads; application of a roughening spray; e.g., titanium plasma spray (TPS); laser blasting; or any other similar process or method.

In a particular embodiment, after installation, the superior compliant layer 520 can be in direct contact with vertebral bone, e.g., cortical bone and cancellous bone. In a particular embodiment, the superior compliant layer 520 can be an extended use biocompatible material. For example, the extended use biocompatible materials can include synthetic polymers, natural polymers, bioactive ceramics, compression molded carbon nanofibers, or combinations thereof.

In a particular embodiment, the synthetic polymers can include polyurethane materials, polyolefin materials, polyether materials, polyester materials, polycarbonate materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the superior compliant layer 520 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 4 through FIG. 7 indicate that the superior component 500 can include a superior keel 548 that extends from superior bearing surface 506. During installation, described below, the superior keel 548 can at least partially engage a keel groove that can be established within a cortical rim of a vertebra. Further, the superior keel 548 can be coated with a bone-growth promoting substance, e.g., a hydroxyapatite coating formed of calcium phosphate. Additionally, the superior bearing surface 506 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

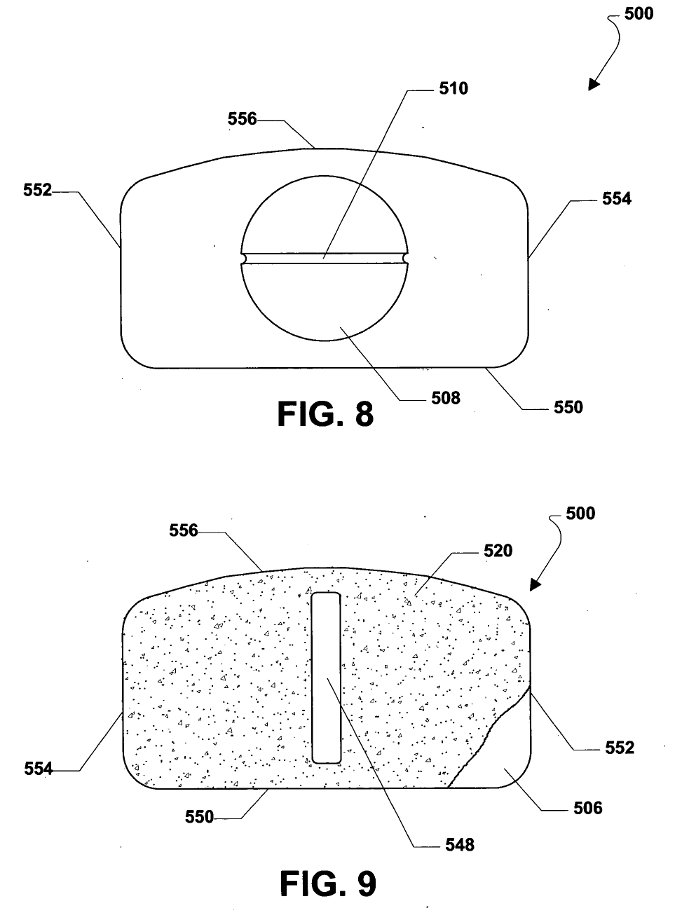

As illustrated in FIG. 8 and FIG. 9, the superior component 500 can be generally rectangular in shape. For example, the superior component 500 can have a substantially straight posterior side 550. A first straight lateral side 552 and a second substantially straight lateral side 554 can extend substantially perpendicular from the posterior side 550 to an anterior side 556. In a particular embodiment, the anterior side 556 can curve outward such that the superior component 500 is wider through the middle than along the lateral sides 552, 554. Further, in a particular embodiment, the lateral sides 552, 554 are substantially the same length.

FIG. 4 and FIG. 5 show that the superior component 500 includes a first implant inserter engagement hole 560 and a second implant inserter engagement hole 562. In a particular embodiment, the implant inserter engagement holes 560, 562 are configured to receive respective dowels, or pins, that extend from an implant inserter (not shown) that can be used to facilitate the proper installation of an intervertebral prosthetic disc, e.g., the intervertebral prosthetic disc 400 shown in FIG. 4 through FIG. 11.

In a particular embodiment, the inferior component 600 includes an inferior support plate 602 that has an inferior articular surface 604 and an inferior bearing surface 606. In a particular embodiment, the inferior articular surface 604 can be generally curved and the inferior bearing surface 606 can be substantially flat. In an alternative embodiment, the inferior articular surface 604 can be substantially flat and at least a portion of the inferior bearing surface 606 can be generally curved.

As illustrated in FIG. 4 through FIG. 7, a depression 608 extends into the inferior articular surface 604 of the inferior support plate 602. In a particular embodiment, the depression 608 is sized and shaped to receive the projection 508 of the superior component 500. For example, the depression 608 can have a hemi-spherical shape. Alternatively, the depression 608 can have an elliptical shape, a cylindrical shape, or other arcuate shape.

As further illustrated, the inferior component 600 includes an inferior compliant layer 620 that can be affixed to, attached to, or otherwise deposited on, the inferior bearing surface 606. The inferior compliant layer 620 can be chemically bonded to the inferior bearing surface 606, e.g., using an adhesive or another chemical bonding agent. Further, the inferior compliant layer 620 can be mechanically anchored to the inferior bearing surface 606, e.g., using hook-and-loop fasteners, or another type of fastener.

Before the inferior compliant layer 620 is deposited, or otherwise affixed to the inferior bearing surface 606, the inferior bearing surface 606 can be modified to promote adhesion of the inferior compliant layer 620 to the inferior bearing surface 606. For example, the inferior bearing surface 606 can be roughened to promote adhesion of the inferior compliant layer 620. For example, the roughening process can include acid etching; knurling; application of a bead coating, e.g., cobalt chrome beads; application of a roughening spray; e.g., titanium plasma spray (TPS); laser blasting; or any other similar process or method.

In a particular embodiment, after installation, the inferior compliant layer 620 can be in direct contact with vertebral bone, e.g., cortical bone and cancellous bone. In a particular embodiment, the inferior compliant layer 620 can be an extended use biocompatible material. For example, the extended use biocompatible materials can include synthetic polymers, natural polymers, bioactive ceramics, compression molded carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the inferior compliant layer 620 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 4 through FIG. 7 indicate that the inferior component 600 can include an inferior keel 648 that extends from inferior bearing surface 606. During installation, described below, the inferior keel 648 can at least partially engage a keel groove that can be established within a cortical rim of a vertebra, e.g., the keel groove 70 shown in FIG. 3. Further, the inferior keel 648 can be coated with a bone-growth promoting substance, e.g., a hydroxyapatite coating formed of calcium phosphate. Additionally, the inferior bearing surface 606 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

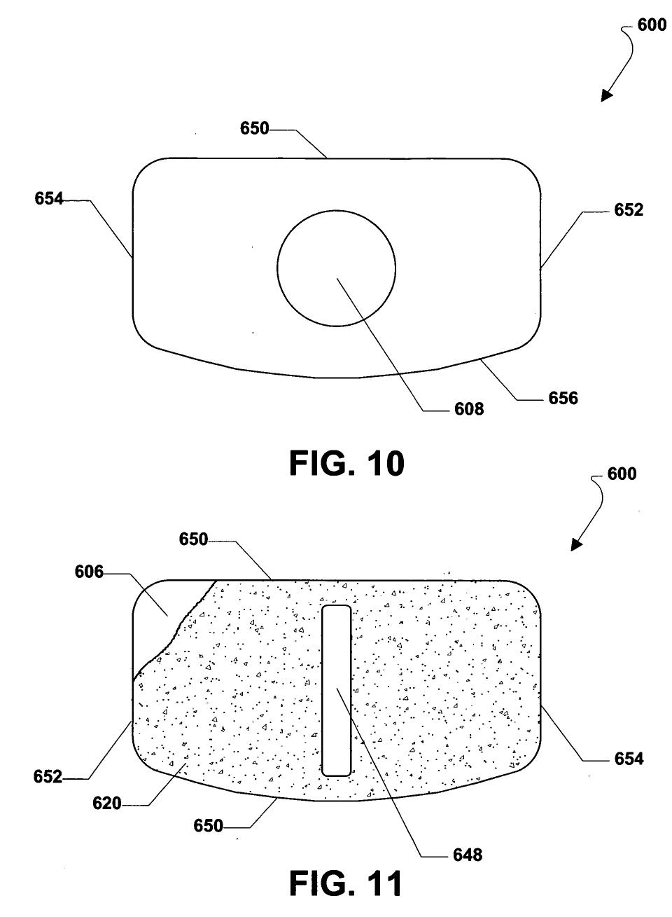

In a particular embodiment, as shown in FIG. 10 and FIG. 11, the inferior component 600 can be shaped to match the shape of the superior component 500, shown in FIG. 8 and FIG. 9. Further, the inferior component 600 can be generally rectangular in shape. For example, the inferior component 600 can have a substantially straight posterior side 650. A first straight lateral side 652 and a second substantially straight lateral side 654 can extend substantially perpendicular from the posterior side 650 to an anterior side 656. In a particular embodiment, the anterior side 656 can curve outward such that the inferior component 600 is wider through the middle than along the lateral sides 652, 654. Further, in a particular embodiment, the lateral sides 652, 654 are substantially the same length.

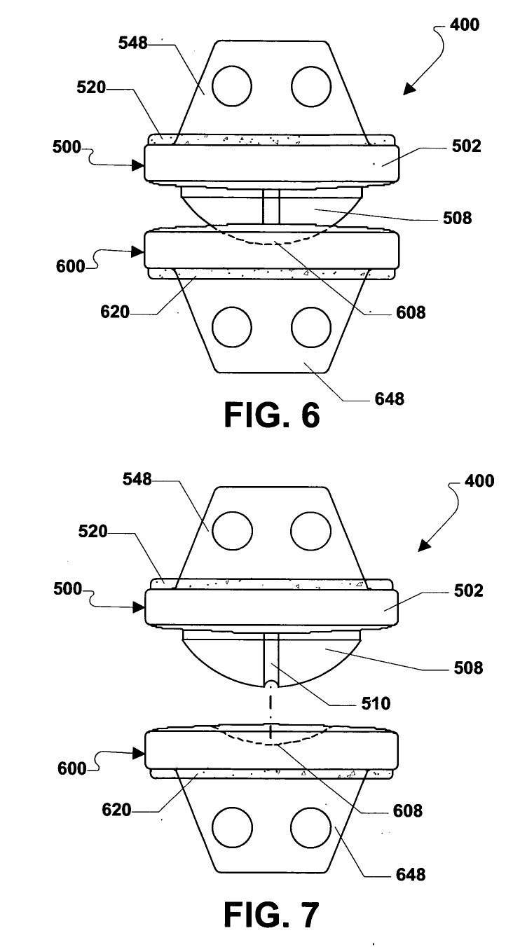

FIG. 4 and FIG. 6 show that the inferior component 600 includes a first implant inserter engagement hole 660 and a second implant inserter engagement hole 662. In a particular embodiment, the implant inserter engagement holes 660, 662 are configured to receive respective dowels, or pins, that extend from an implant inserter (not shown) that can be used to facilitate the proper installation of an intervertebral prosthetic disc, e.g., the intervertebral prosthetic disc 400 shown in FIG. 4 through FIG. 9.

In a particular embodiment, the overall height of the intervertebral prosthetic device 400 can be in a range from fourteen millimeters to forty-six millimeters (14-46 mm). Further, the installed height of the intervertebral prosthetic device 400 can be in a range from eight millimeters to sixteen millimeters (8-16 mm). In a particular embodiment, the installed height can be substantially equivalent to the distance between an inferior vertebra and a superior vertebra when the intervertebral prosthetic device 400 is installed there between.

In a particular embodiment, the length of the intervertebral prosthetic device 400, e.g., along a longitudinal axis, can be in a range from thirty millimeters to forty millimeters (30-40 mm). Additionally, the width of the intervertebral prosthetic device 400, e.g., along a lateral axis, can be in a range from twenty-five millimeters to forty millimeters (25-40 mm). Moreover, in a particular embodiment, each keel 548, 648 can have a height in a range from three millimeters to fifteen millimeters (3-15 mm). Installation of the First Embodiment within an Intervertebral Space

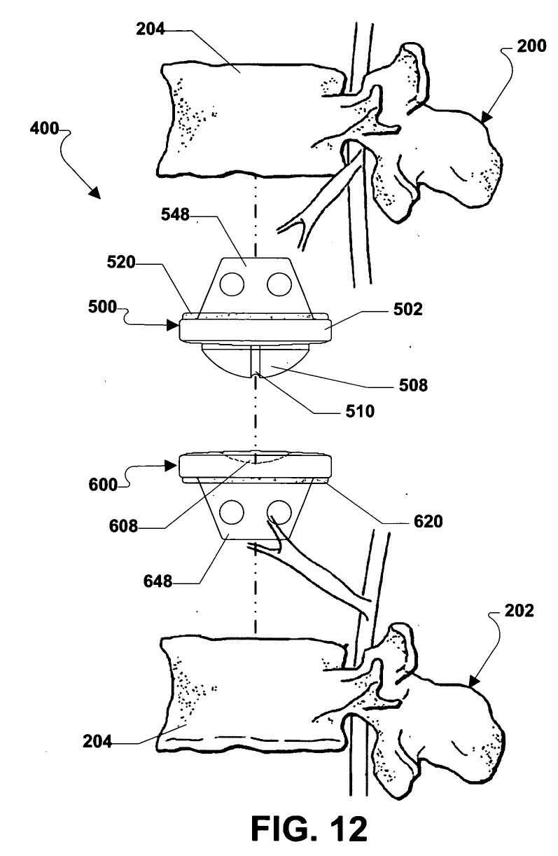

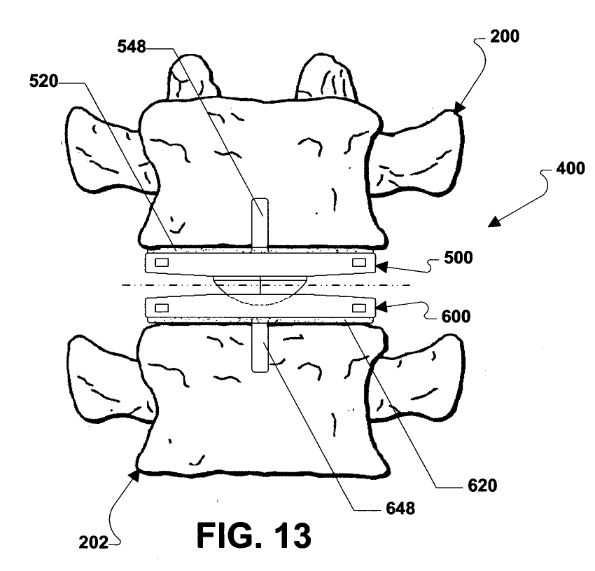

Referring to FIG. 12 and FIG. 13, an intervertebral prosthetic disc is shown between the superior vertebra 200 and the inferior vertebra 202, previously introduced and described in conjunction with FIG. 2. In a particular embodiment, the intervertebral prosthetic disc is the intervertebral prosthetic disc 400 described in conjunction with FIG. 4 through FIG. 11. Alternatively, the intervertebral prosthetic disc can be an intervertebral prosthetic disc according to any of the embodiments disclosed herein.

As shown in FIG. 12 and FIG. 13, the intervertebral prosthetic disc 400 is installed within the intervertebral space 214 that can be established between the superior vertebra 200 and the inferior vertebra 202 by removing vertebral disc material (not shown). In a particular embodiment, the superior keel 548 of the superior component 500 can at least partially engage the cancellous bone and cortical rim of the superior vertebra 200. Also, in a particular embodiment, the inferior keel 648 of the inferior component 600 can at least partially engage the cancellous bone and cortical rim of the inferior vertebra 202.

FIG. 13 indicates that the superior compliant layer 520 can engage the superior vertebra 200, e.g., the cortical rim and cancellous bone of the superior vertebra 200. The superior compliant layer 520 can mold, or otherwise form, to match the shape of the cortical rim and cancellous bone of the superior vertebra 200. In a particular embodiment, the superior compliant layer 520 can increase the contact area between the superior vertebra 200 and the superior support plate 502. As such, the superior compliant layer 520 can substantially reduce the contact stress between the superior vertebra 200 and the superior support plate 502.

Also, the inferior compliant layer 620 can engage the inferior vertebra 202, e.g., the cortical rim and cancellous bone of the inferior vertebra 202. The inferior compliant layer 620 can mold, or otherwise form, to match the shape of the cortical rim and cancellous bone of the inferior vertebra 200. In a particular embodiment, the inferior compliant layer 620 can increase the contact area between the inferior vertebra 200 and the inferior support plate 602. As such, the inferior compliant layer 620 can substantially reduce the contact stress between the inferior vertebra 200 and the inferior support plate 602.

As illustrated in FIG. 12 and FIG. 13, the projection 508 that extends from the superior component 500, of the intervertebral prosthetic disc 400 can at least partially engage the depression 608 that is formed within the inferior component 600 of the intervertebral prosthetic disc 400. It is to be appreciated that when the intervertebral prosthetic disc 400 is installed between the superior vertebra 200 and the inferior vertebra 202, the intervertebral prosthetic disc 400 allows relative motion between the superior vertebra 200 and the inferior vertebra 202. Specifically, the configuration of the superior component 500 and the inferior component 600 allows the superior component 500 to rotate with respect to the inferior component 600. As such, the superior vertebra 200 can rotate with respect to the inferior vertebra 202.

In a particular embodiment, the intervertebral prosthetic disc 400 can allow angular movement in any radial direction relative to the intervertebral prosthetic disc 400. Further, as depicted in FIG. 13, the inferior component 600 can be placed on the inferior vertebra 202 so that the center of rotation of the inferior component 600 is substantially aligned with the center of rotation of the inferior vertebra 202. Similarly, the superior component 500 can be placed relative to the superior vertebra 200 so that the center of rotation of the superior component 500 is substantially aligned with the center of rotation of the superior vertebra 200. Accordingly, when the vertebral disc, between the inferior vertebra 202 and the superior vertebra 200, is removed and replaced with the intervertebral prosthetic disc 400 the relative motion of the vertebrae 200, 202 provided by the vertebral disc is substantially replicated.

Description of a Second Embodiment of an Intervertebral Prosthetic Disc

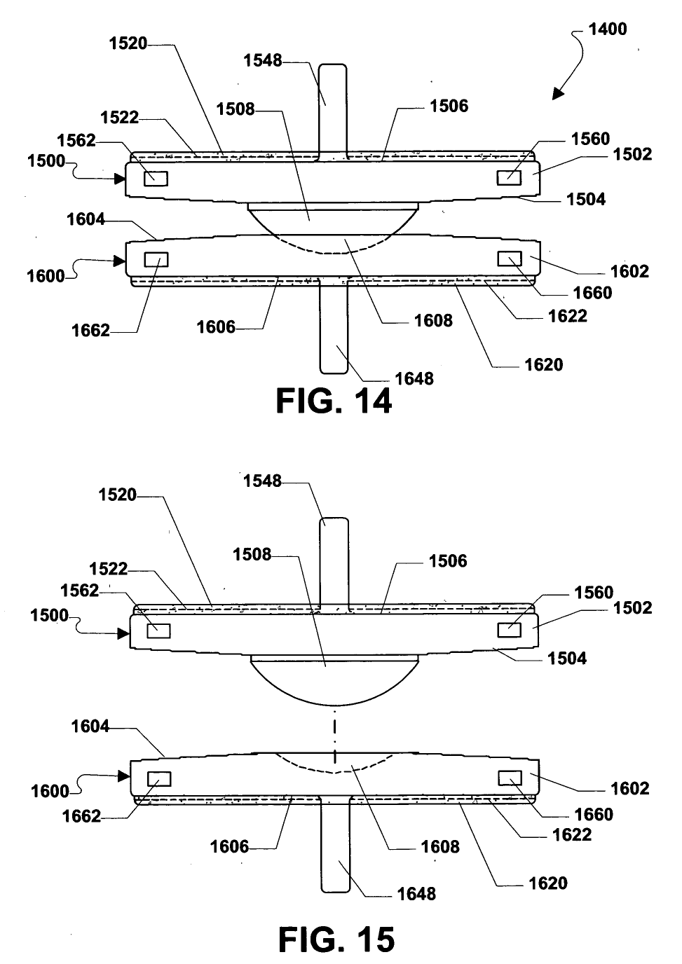

Referring to FIGS. 14 through 21 a first embodiment of an intervertebral prosthetic disc is shown and is generally designated 1400. As illustrated, the intervertebral prosthetic disc 1400 includes a superior component 1500 and an inferior component 1600. In a particular embodiment, the components 1500, 1600 can be made from one or more extended use biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 1500, 1600 can be made from any other substantially rigid biocompatible materials.

In a particular embodiment, the superior component 1500 includes a superior support plate 1502 that has a superior articular surface 1504 and a superior bearing surface 1506. In a particular embodiment, the superior articular surface 1504 can be generally curved and the superior bearing surface 1506 can be substantially flat. In an alternative embodiment, the superior articular surface 1504 can be substantially flat and at least a portion of the superior bearing surface 1506 can be generally curved.

As illustrated in FIG. 14 through FIG. 17, a projection 1508 extends from the superior articular surface 1504 of the superior support plate 1502. In a particular embodiment, the projection 1508 has a hemi-spherical shape. Alternatively, the projection 1508 can have an elliptical shape, a cylindrical shape, or other arcuate shape. Moreover, the projection 1508 can be formed with a groove 1510.

As further illustrated, the superior component 1500 includes a superior compliant layer 1520 that can be affixed to, attached to, or otherwise deposited on, the superior bearing surface 1506. The superior compliant layer 1520 can be chemically bonded to the superior bearing surface 1506, e.g., using an adhesive or another chemical bonding agent. Further, the superior compliant layer 1520 can be mechanically anchored to the superior bearing surface 1506, e.g., using hook-and-loop fasteners, or another type of fastener.

Before the superior compliant layer 1520 is deposited, or otherwise affixed to the superior bearing surface 1506, the superior bearing surface 1506 can be modified to promote adhesion of the superior compliant layer 1520 to the superior bearing surface 1506. For example, the superior bearing surface 1506 can be roughened to promote adhesion of the superior compliant layer 1520. For example, the roughening process can include acid etching; knurling; application of a bead coating, e.g., cobalt chrome beads; application of a roughening spray; e.g., titanium plasma spray (TPS); laser blasting; or any other similar process or method.

In a particular embodiment, after installation, the superior compliant layer 1520 can be in direct contact with vertebral bone, e.g., cortical bone and cancellous bone. In a particular embodiment, the superior compliant layer 1520 can be an extended use biocompatible material. For example, the extended use biocompatible materials can include synthetic polymers, natural polymers, bioactive ceramics, compression molded carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the superior compliant layer 1520 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

As indicated in FIG. 14 through FIG. 17 and FIG. 19 a superior embedded structure 1522 can be disposed, implanted, embedded, or otherwise suspended, within the superior compliant surface 1520. The superior embedded structure 1522 can be a fabric mesh, a metallic mesh, a PEEK mesh, a three dimensional (3-D) polyester embedded structure, or a combination thereof. Further, the embedded structure 1522 can be non-resorbable while the superior compliant surface 1520 is resorbable. As such, the superior compliant surface 1520 can be resorbed as bone grows onto the superior component 1500 and the bone can penetrate the non-resorbable mesh.

FIG. 14 through FIG. 17 indicate that the superior component 1500 can include a superior keel 1548 that extends from superior bearing surface 1506. During installation, described below, the superior keel 1548 can at least partially engage a keel groove that can be established within a cortical rim of a vertebra. Further, the superior keel 1548 can be coated with a bone-growth promoting substance, e.g., a hydroxyapatite coating formed of calcium phosphate. Additionally, the superior bearing surface 1506 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

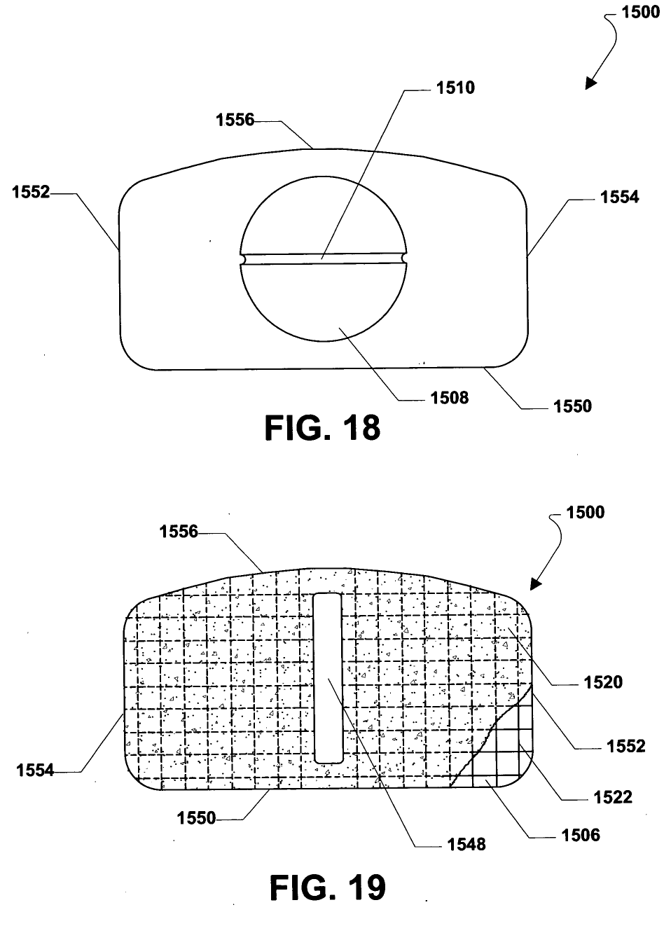

As illustrated in FIG. 18 and FIG. 19, the superior component 1500 can be generally rectangular in shape. For example, the superior component 1500 can have a substantially straight posterior side 1550. A first straight lateral side 1552 and a second substantially straight lateral side 1554 can extend substantially perpendicular from the posterior side 1550 to an anterior side 1556. In a particular embodiment, the anterior side 1556 can curve outward such that the superior component 1500 is wider through the middle than along the lateral sides 1552, 1554. Further, in a particular embodiment, the lateral sides 1552, 1554 are substantially the same length.

FIG. 14 and FIG. 15 show that the superior component 1500 includes a first implant inserter engagement hole 1560 and a second implant inserter engagement hole 1562. In a particular embodiment, the implant inserter engagement holes 1560, 1562 are configured to receive respective dowels, or pins, that extend from an implant inserter (not shown) that can be used to facilitate the proper installation of an intervertebral prosthetic disc, e.g., the intervertebral prosthetic disc 1400 shown in FIG. 14 through FIG. 21.

In a particular embodiment, the inferior component 1600 includes an inferior support plate 1602 that has an inferior articular surface 1604 and an inferior bearing surface 1606. In a particular embodiment, the inferior articular surface 1604 can be generally curved and the inferior bearing surface 1606 can be substantially flat. In an alternative embodiment, the inferior articular surface 1604 can be substantially flat and at least a portion of the inferior bearing surface 1606 can be generally curved.

As illustrated in FIG. 14 through FIG. 17, a depression 1608 extends into the inferior articular surface 1604 of the inferior support plate 1602. In a particular embodiment, the depression 1608 is sized and shaped to receive the projection 1508 of the superior component 1500. For example, the depression 1608 can have a hemi-spherical shape. Alternatively, the depression 1608 can have an elliptical shape, a cylindrical shape, or other arcuate shape.

As further illustrated, the inferior component 1600 includes an inferior compliant layer 1620 that can be affixed to, attached to, or otherwise deposited on, the inferior bearing surface 1606. The inferior compliant layer 1620 can be chemically bonded to the inferior bearing surface 1606, e.g., using an adhesive or another chemical bonding agent. Further, the inferior compliant layer 1620 can be mechanically anchored to the inferior bearing surface 1606, e.g., using hook-and-loop fasteners, or another type of fastener.

Before the inferior compliant layer 1620 is deposited, or otherwise affixed to the inferior bearing surface 1606, the inferior bearing surface 1606 can be modified to promote adhesion of the inferior compliant layer 1620 to the inferior bearing surface 1606. For example, the inferior bearing surface 1606 can be roughened to promote adhesion of the inferior compliant layer 1620. For example, the roughening process can include acid etching; knurling; application of a bead coating, e.g., cobalt chrome beads; application of a roughening spray; e.g., titanium plasma spray (TPS); laser blasting; or any other similar process or method.

In a particular embodiment, after installation, the inferior compliant layer 1620 can be in direct contact with vertebral bone, e.g., cortical bone and cancellous bone. In a particular embodiment, the inferior compliant layer 1620 can be an extended use biocompatible material. For example, the extended use biocompatible materials can include synthetic polymers, natural polymers, bioactive ceramics, compression molded carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the inferior compliant layer 1620 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

As indicated in FIG. 14 through FIG. 17 and FIG. 21 an inferior embedded structure 1622 can be disposed, implanted, embedded, or otherwise suspended within the inferior compliant surface 1620. The inferior embedded structure 1622 can be a fabric mesh, a metallic mesh, a PEEK mesh, a three dimensional (3-D) polyester structure, or a combination thereof. Further, the embedded structure 1622 can be non-resorbable while the inferior compliant surface 1620 is resorbable. As such, the inferior compliant surface 1620 can be resorbed as bone grows onto the inferior component 1600 and the bone can penetrate the non-resorbable mesh.

FIG. 14 through FIG. 17 indicate that the inferior component 1600 can include an inferior keel 1648 that extends from inferior bearing surface 1606. During installation, described below, the inferior keel 1648 can at least partially engage a keel groove that can be established within a cortical rim of a vertebra. Further, the inferior keel 1648 can be coated with a bone-growth promoting substance, e.g., a hydroxyapatite coating formed of calcium phosphate. Additionally, the inferior bearing surface 1606 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

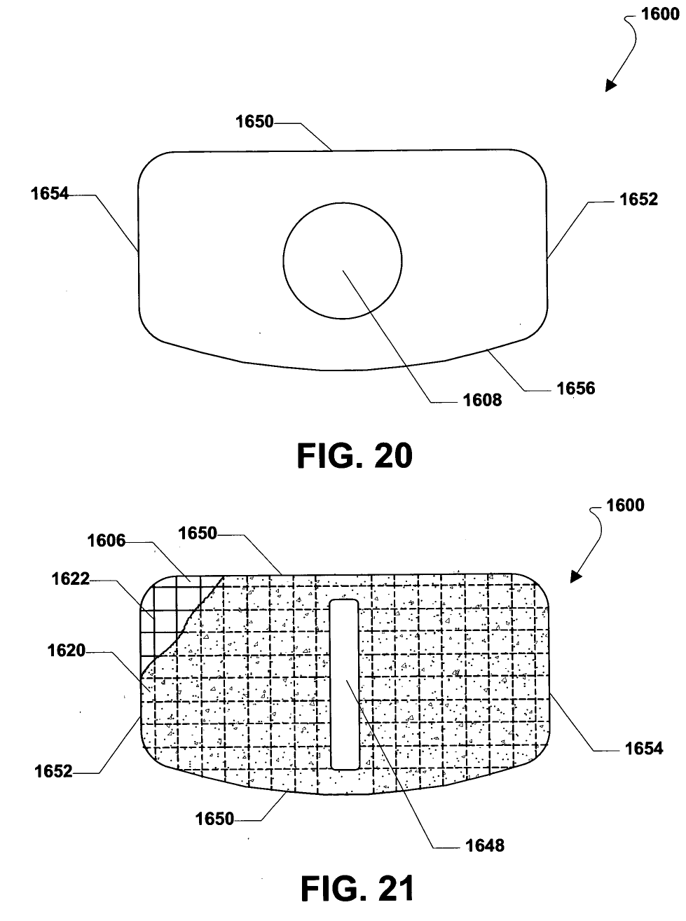

In a particular embodiment, as shown in FIG. 20 and FIG. 21, the inferior component 1600 can be shaped to match the shape of the superior component 1500, shown in FIG. 18 and FIG. 19. Further, the inferior component 1600 can be generally rectangular in shape. For example, the inferior component 1600 can have a substantially straight posterior side 1650. A first straight lateral side 1652 and a second substantially straight lateral side 1654 can extend substantially perpendicular from the posterior side 1650 to an anterior side 1656. In a particular embodiment, the anterior side 1656 can curve outward such that the inferior component 1600 is wider through the middle than along the lateral sides 1652, 1654. Further, in a particular embodiment, the lateral sides 1652, 1654 are substantially the same length.

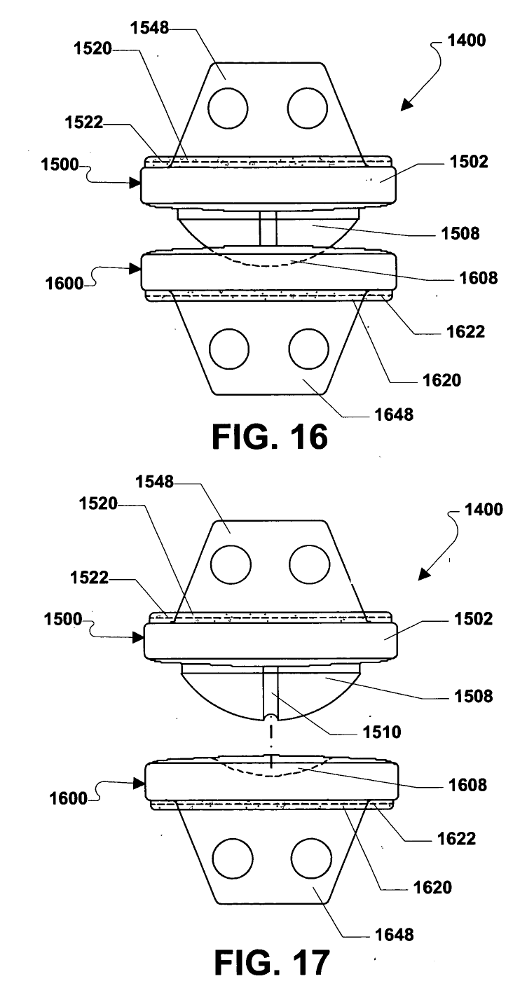

FIG. 14 and FIG. 16 show that the inferior component 1600 includes a first implant inserter engagement hole 1660 and a second implant inserter engagement hole 1662. In a particular embodiment, the implant inserter engagement holes 1660, 1662 are configured to receive respective dowels, or pins, that extend from an implant inserter (not shown) that can be used to facilitate the proper installation of an intervertebral prosthetic disc, e.g., the intervertebral prosthetic disc 1400 shown in FIG. 14 through FIG. 19.

In a particular embodiment, the overall height of the intervertebral prosthetic device 1400 can be in a range from fourteen millimeters to forty-six millimeters (14-46 mm). Further, the installed height of the intervertebral prosthetic device 1400 can be in a range from eight millimeters to sixteen millimeters (8-16 mm). In a particular embodiment, the installed height can be substantially equivalent to the distance between an inferior vertebra and a superior vertebra when the intervertebral prosthetic device 1400 is installed there between.

In a particular embodiment, the length of the intervertebral prosthetic device 1400, e.g., along a longitudinal axis, can be in a range from thirty millimeters to forty millimeters (30-40 mm). Additionally, the width of the intervertebral prosthetic device 1400, e.g., along a lateral axis, can be in a range from twenty-five millimeters to forty millimeters (25-40 mm). Moreover, in a particular embodiment, each keel 1548, 1648 can have a height in a range from three millimeters to fifteen millimeters (3-15 mm).

Description of a Third Embodiment of an Intervertebral Prosthetic Disc

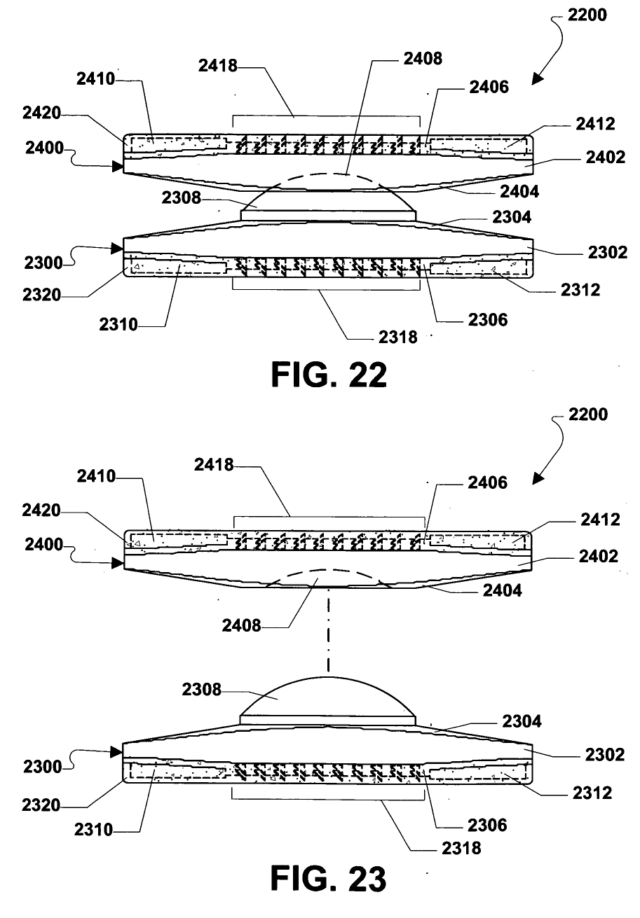

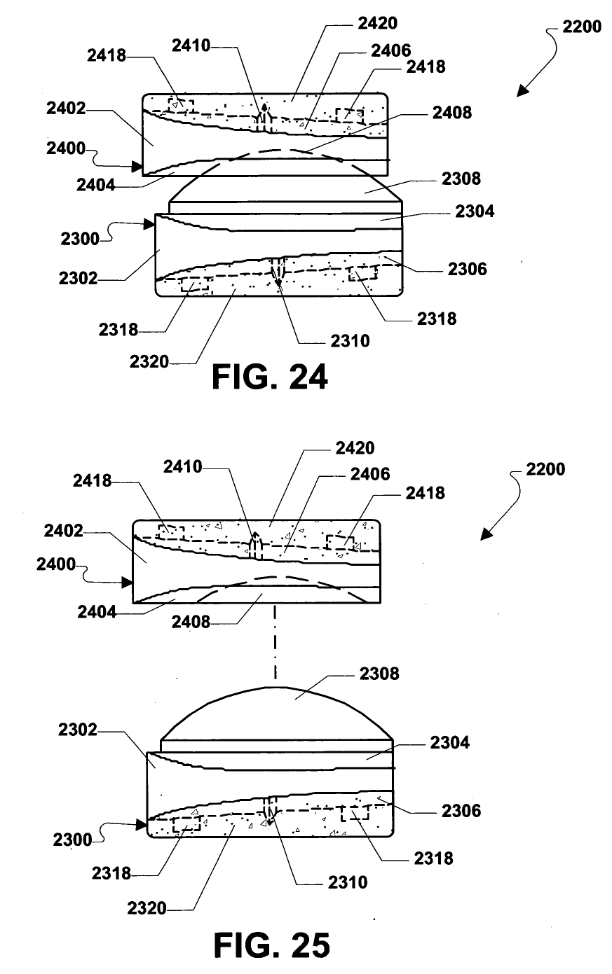

Referring to FIGS. 22 through 29 a third embodiment of an intervertebral prosthetic disc is shown and is generally designated 2200. As illustrated, the intervertebral prosthetic disc 2200 includes an inferior component 2300 and a superior component 2400. In a particular embodiment, the components 2300, 2400 can be made from one or more extended use biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 2300, 2400 can be made from any other substantially rigid biocompatible materials.

In a particular embodiment, the inferior component 2300 includes an inferior support plate 2302 that has an inferior articular surface 2304 and an inferior bearing surface 2306. In a particular embodiment, the inferior articular surface 2304 and the inferior bearing surface 2306 are generally rounded.

As illustrated in FIG. 22 through FIG. 29, a projection 2308 extends from the inferior articular surface 2304 of the inferior support plate 2302. In a particular embodiment, the projection 2308 has a hemi-spherical shape. Alternatively, the projection 2308 can have an elliptical shape, a cylindrical shape, or other arcuate shape.

As further illustrated in FIG. 22 through FIG. 25 and FIG. 27, the inferior component 2300 includes a first inferior keel 2310 and a second inferior keel 2312 that extend substantially perpendicularly from the inferior bearing surface 2306. In a particular embodiment, as shown in FIG. 27, the first inferior keel 2310 and the second inferior keel 2312 extend along a longitudinal axis 2314 defined by the inferior component 2300. As shown, the first inferior keel 2310 and the second inferior keel 2312 can extend along the longitudinal axis 2314 from a perimeter of the inferior component 2300 toward a lateral axis 2316 that is defined by the inferior component 2300. In a particular embodiment, the first inferior keel 2310 and the second inferior keel 2312 are sized and shaped to engage a first and second keel groove that can be established within a cortical rim of an inferior vertebra.

FIG. 22 through FIG. 25 and FIG. 27 also show that the inferior component 2300 includes a plurality of inferior teeth 2318 that extend from the inferior bearing surface 2306. As shown, in a particular embodiment, the inferior teeth 2318 are generally saw-tooth, or triangle, shaped. Further, the inferior teeth 2318 are designed to engage cancellous bone of an inferior vertebra. Additionally, the inferior teeth 2318 can prevent the inferior component 2300 from moving with respect to an inferior vertebra after the intervertebral prosthetic disc 2200 is installed within the intervertebral space between the inferior vertebra and the superior vertebra.

In a particular embodiment, the inferior teeth 2318 can include other projections such as spikes, pins, blades, or a combination thereof that have any cross-sectional geometry.

As illustrated in FIG. 22 through FIG. 25 and FIG. 27, the inferior component 2300 can further include an inferior compliant layer 2320 that can be affixed to, attached to, or otherwise deposited on, the inferior bearing surface 2306. The inferior compliant layer 2320 can be chemically bonded to the inferior bearing surface 2306, e.g., using an adhesive or another chemical bonding agent. Further, the inferior compliant layer 2320 can be mechanically anchored to the inferior bearing surface 2306, e.g., using hook-and-loop fasteners, or another type of fastener.

As shown, the inferior compliant layer 2320 can at least partially cover the inferior keels 2310, 2312 and the inferior teeth 2318. Accordingly, when the intervertebral prosthetic disc 2200 is implanted in a patient, the inferior compliant layer 2320 can compress and comply with the shape of a vertebra. Further, as the inferior compliant layer 2320 compresses, the inferior keels 2310, 2312 and the inferior teeth 2318 can at least partially engage cortical bone of the vertebra, cancellous bone of the vertebra, or a combination thereof.

Before the inferior compliant layer 2320 is deposited, or otherwise affixed to the inferior bearing surface 2306, the inferior bearing surface 2306 can be modified to promote adhesion of the inferior compliant layer 2320 to the inferior bearing surface 2306. For example, the inferior bearing surface 2306 can be roughened to promote adhesion of the inferior compliant layer 2320. For example, the roughening process can include acid etching; knurling; application of a bead coating, e.g., cobalt chrome beads; application of a roughening spray; e.g., titanium plasma spray (TPS); laser blasting; or any other similar process or method.

In a particular embodiment, the inferior compliant layer 2320 can be an extended use biocompatible material. For example, the extended use biocompatible materials can include synthetic polymers, natural polymers, bioactive ceramics, compression molded carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the inferior compliant layer 2320 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

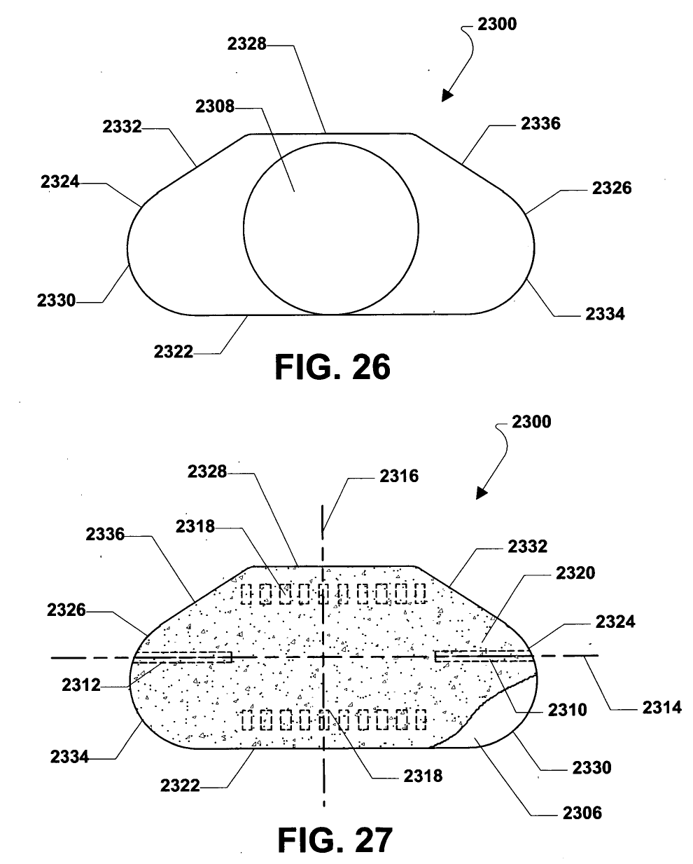

As illustrated in FIG. 26 and FIG. 27, the inferior component 2300 can be generally shaped to match the general shape of the vertebral body of a vertebra. For example, the inferior component 2300 can have a general trapezoid shape and the inferior component 2300 can include a posterior side 2322. A first lateral side 2324 and a second lateral side 2326 can extend from the posterior side 2322 to an anterior side 2328. In a particular embodiment, the first lateral side 2324 includes a curved portion 2330 and a straight portion 2332 that extends at an angle toward the anterior side 2328. Further, the second lateral side 2326 can also include a curved portion 2334 and a straight portion 2336 that extends at an angle toward the anterior side 2328.

As shown in FIG. 26 and FIG. 27, the anterior side 2328 of the inferior component 2300 can be relatively shorter than the posterior side 2322 of the inferior component 2300. Further, in a particular embodiment, the anterior side 2328 is substantially parallel to the posterior side 2322. As indicated in FIG. 26, the projection 2308 can be situated, or otherwise formed, on the inferior articular surface 2304 such that the perimeter of the projection 2308 is tangential to the posterior side 2322 of the inferior component 2300. In alternative embodiments (not shown), the projection 2308 can be situated, or otherwise formed, on the inferior articular surface 2304 such that the perimeter of the projection 2308 is tangential to the anterior side 2328 of the inferior component 2300 or tangential to both the anterior side 2328 and the posterior side 2322. In a particular embodiment, the projection 2308 and the inferior support plate 2302 comprise a monolithic body.

In a particular embodiment, the superior component 2400 includes a superior support plate 2402 that has a superior articular surface 2404 and a superior bearing surface 2406. In a particular embodiment, the superior articular surface 2404 and the superior bearing surface 2406 are generally rounded.

As illustrated in FIG. 22 through FIG. 25 and FIG. 28, a depression 2408 extends into the superior articular surface 2404 of the superior support plate 2402. In a particular embodiment, the depression 2408 is sized and shaped to receive the projection 2308 of the inferior component 2300. For example, the depression 2408 can have a hemi-spherical shape. Alternatively, the depression 2408 can have an elliptical shape, a cylindrical shape, or other arcuate shape.

As further illustrated in FIG. 22 through 25 and FIG. 29, the superior component 2400 includes a first superior keel 2410 and a second superior keel 2412 that extend substantially perpendicularly from the superior bearing surface 2406. In a particular embodiment, the first superior keel 2410 and the second superior keel 2412 of the superior component 2400 are arranged in a manner similar to the first inferior keel 2310 and the second inferior keel 2312 of the inferior component 2300, as shown in FIG. 27. In another particular embodiment, the first superior keel 2410 and the second superior keel 2412 are sized and shaped to engage a first and second keel groove that can be established within a cortical rim of a superior vertebra.

FIG. 22 through FIG. 29 also show that the superior component 2400 includes a plurality of superior teeth 2418 that extend from the superior bearing surface 2406. As shown, in a particular embodiment, the superior teeth 2418 are generally saw-tooth, or triangle, shaped. Further, the superior teeth 2418 are designed to engage cancellous bone, e.g., the cancellous bone 404 of the superior vertebra 302 shown in FIG. 4. Additionally, the superior teeth 2418 can prevent the superior component 2400 from moving with respect to a superior vertebra after the intervertebral prosthetic disc 2200 is installed within an intervertebral space between an inferior vertebra and the superior vertebra.

In a particular embodiment, the superior teeth 2418 can include other projections such as spikes, pins, blades, or a combination thereof that have any cross-sectional geometry.

As illustrated in FIG. 22 through FIG. 25 and FIG. 29, the superior component 2400 can further include a superior compliant layer 2420 that can be affixed to, attached to, or otherwise deposited on, the superior bearing surface 2406. The superior compliant layer 2420 can be chemically bonded to the superior bearing surface 2406, e.g., using an adhesive or another chemical bonding agent. Further, the superior compliant layer 2420 can be mechanically anchored to the superior bearing surface 2406, e.g., using hook-and-loop fasteners, or another type of fastener.

As shown, the superior compliant layer 2420 can at least partially cover the superior keels 2410, 2412 and the superior teeth 2418. Accordingly, when the intervertebral prosthetic disc 2200 is implanted in a patient, the superior compliant layer 2420 can compress and comply with the shape of a vertebra. Further, as the superior compliant layer 2420 compresses, the superior keels 2410, 2412 and the superior teeth 2418 can at least partially engage cortical bone of the vertebra, cancellous bone of the vertebra, or a combination thereof.

Before the superior compliant layer 2420 is deposited, or otherwise affixed to the superior bearing surface 2406, the superior bearing surface 2406 can be modified to promote adhesion of the superior compliant layer 2420 to the superior bearing surface 2406. For example, the superior bearing surface 2406 can be roughened to promote adhesion of the superior compliant layer 2420. For example, the roughening process can include acid etching; knurling; application of a bead coating, e.g., cobalt chrome beads; application of a roughening spray; e.g., titanium plasma spray (TPS); laser blasting; or any other similar process or method.

In a particular embodiment, the superior compliant layer 2420 can be an extended use biocompatible material. For example, the extended use biocompatible materials can include synthetic polymers, natural polymers, bioactive ceramics, compression molded carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the superior compliant layer 2420 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

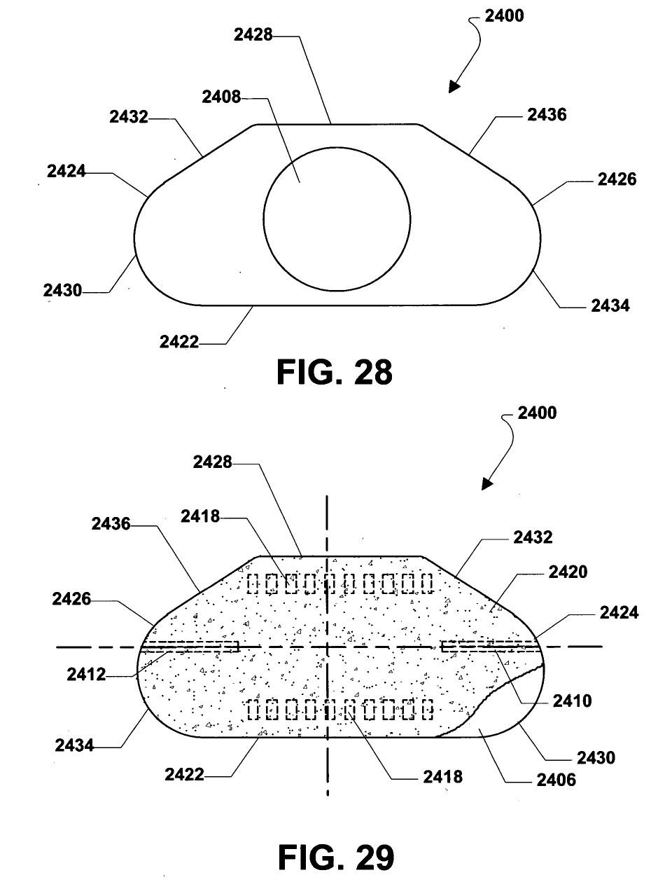

In a particular embodiment, the superior component 2400 can be shaped to match the shape of the inferior component 2300, shown in FIG. 26 and FIG. 27. Further, the superior component 2400 can be shaped to match the general shape of a vertebral body of a vertebra. For example, as shown in FIG. 28 and FIG. 29, the superior component 2400 can have a general trapezoid shape and the superior component 2400 can include a posterior side 2422. A first lateral side 2424 and a second lateral side 2426 can extend from the posterior side 2422 to an anterior side 2428. In a particular embodiment, the first lateral side 2424 includes a curved portion 2430 and a straight portion 2432 that extends at an angle toward the anterior side 2428. Further, the second lateral side 2426 can also include a curved portion 2434 and a straight portion 2436 that extends at an angle toward the anterior side 2428.

As shown in FIG. 28 and FIG. 29, the anterior side 2428 of the superior component 2400 can be relatively shorter than the posterior side 2422 of the superior component 2400. Further, in a particular embodiment, the anterior side 2428 is substantially parallel to the posterior side 2422.

In a particular embodiment, the overall height of the intervertebral prosthetic device 2200 can be in a range from six millimeters to twenty-two millimeters (6-22 mm). Further, the installed height of the intervertebral prosthetic device 2200 can be in a range from four millimeters to sixteen millimeters (4-16 mm). In a particular embodiment, the installed height can be substantially equivalent to the distance between an inferior vertebra and a superior vertebra when the intervertebral prosthetic device 2200 is installed there between.

In a particular embodiment, the length of the intervertebral prosthetic device 2200, e.g., along a longitudinal axis, can be in a range from thirty-three millimeters to fifty millimeters (33-50 mm). Additionally, the width of the intervertebral prosthetic device 2200, e.g., along a lateral axis, can be in a range from eighteen millimeters to twenty-nine millimeters (18-29 mm). Moreover, in a particular embodiment, each keel 2310, 2312, 2410, 2412 can have a height in a range from one millimeter to six millimeters (1-6 mm). In a particular embodiment, the height of each keel 2310, 2312, 2410, 2412 is measured at a location of each keel 2310, 2312, 2410, 2412 nearest to the center of each half 2300, 2400 of the intervertebral prosthetic device 2200.

In a particular embodiment, the keels 2310, 2312, 2410, 2412 can be considered “low profile”. Further, intervertebral prosthetic disc 2200 can be considered to be “low profile.” The low profile of the keels 2310, 2312, 2410, 2412 and the intervertebral prosthetic device 2200 can allow the intervertebral prosthetic device 2200 to be implanted into an intervertebral space between an inferior vertebra and a superior vertebra laterally through a patient's psoas muscle, e.g., through an insertion device. Accordingly, the risk of damage to a patient's spinal cord or sympathetic chain can be substantially minimized. In alternative embodiments, all of the superior and inferior teeth 2318, 2418 can be oriented to engage in a direction substantially opposite the direction of insertion of the prosthetic disc into the intervertebral space.

Further, the intervertebral prosthetic disc 2200 can have a general “bullet” shape as shown in the posterior plan view, described herein. The bullet shape of the intervertebral prosthetic disc 2200 provided by the rounded bearing surfaces 2304, 2404 can further allow the intervertebral prosthetic disc 2200 to be inserted through the patient's psoas muscle while minimizing risk to the patient's spinal cord and sympathetic chain.

Description of a Fourth Embodiment of an Intervertebral Prosthetic Disc

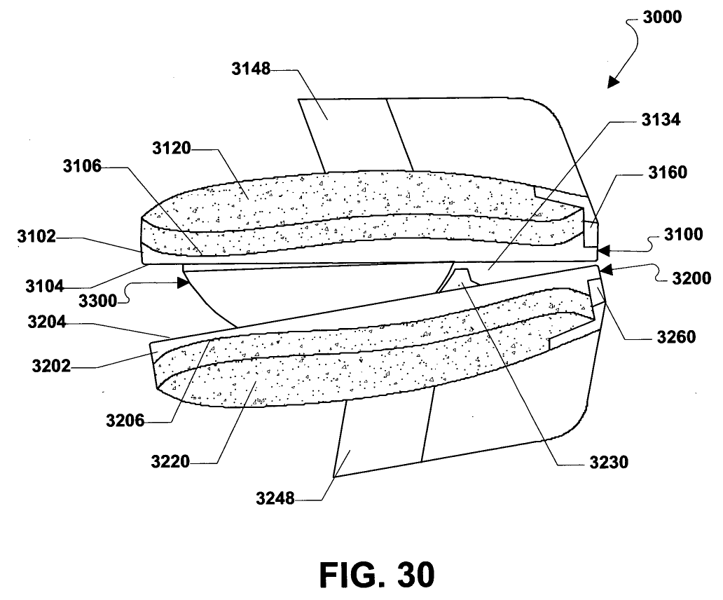

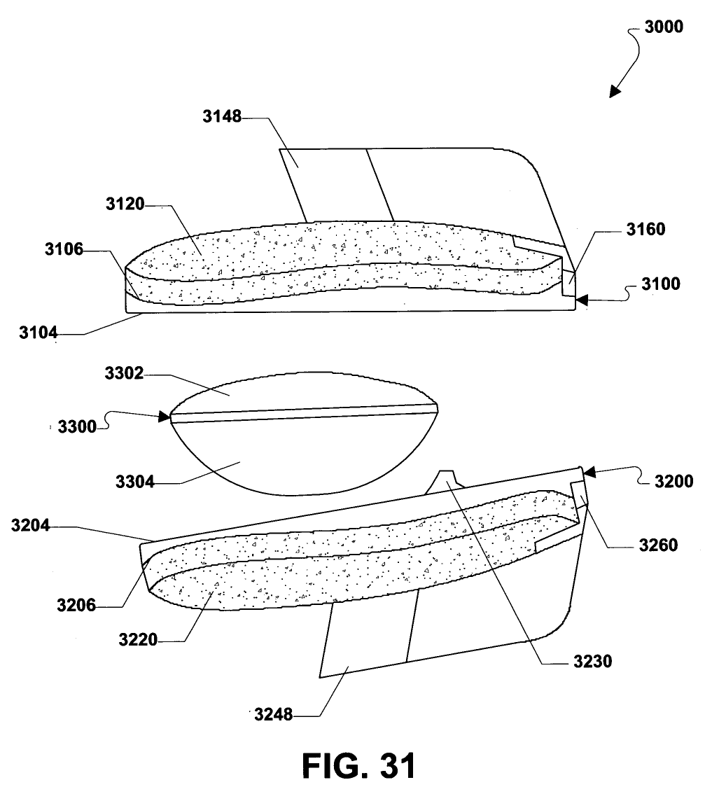

Referring to FIGS. 30 through 34 a fourth embodiment of an intervertebral prosthetic disc is shown and is generally designated 3000. As illustrated, the intervertebral prosthetic disc 3000 includes a superior component 3100, an inferior component 3200, and a nucleus 3300 disposed, or otherwise installed, there between. In a particular embodiment, the components 3100, 3200 and the nucleus 3300 can be made from one or more extended use biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 3100, 3200 can be made from any other substantially rigid biocompatible materials.

In a particular embodiment, the superior component 3100 includes a superior support plate 3102 that has a superior articular surface 3104 and a superior bearing surface 3106. In a particular embodiment, the superior articular surface 3104 can be substantially flat and the superior bearing surface 3106 can be generally curved. In an alternative embodiment, at least a portion of the superior articular surface 3104 can be generally curved and the superior bearing surface 3106 can be substantially flat.

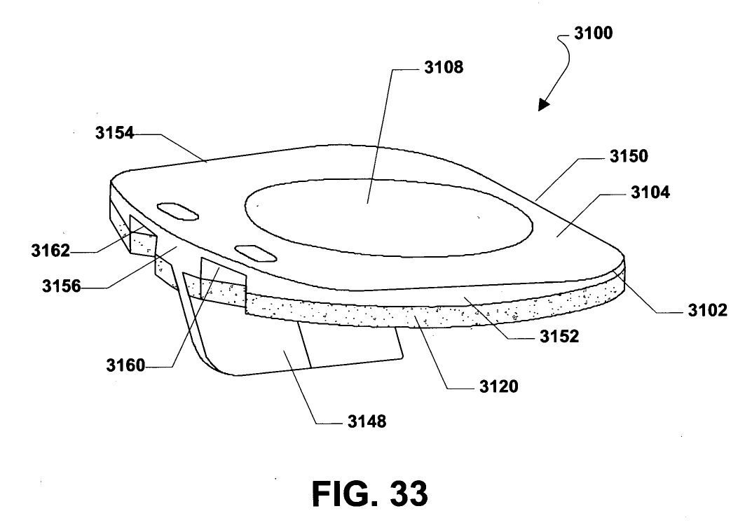

As illustrated in FIG. 33, a superior depression 3108 is established within the superior articular surface 3104 of the superior support plate 3102. In a particular embodiment, the superior depression 3108 has an arcuate shape. For example, the superior depression 3108 can have a hemispherical shape, an elliptical shape, a cylindrical shape, or any combination thereof.

As further illustrated, the superior component 3100 includes a superior compliant layer 3120 that can be affixed to, attached to, or otherwise deposited on, the superior bearing surface 3106. As shown, the superior compliant layer 3120 can be substantially convex. Further, the superior compliant layer 3120 can have a thickness that is substantially uniform. Alternatively, the superior compliant layer 3120 can have a thickness that varies throughout the superior compliant layer 3120.

The superior compliant layer 3120 can be chemically bonded to the superior bearing surface 3106, e.g., using an adhesive or another chemical bonding agent. Further, the superior compliant layer 3120 can be mechanically anchored to the superior bearing surface 3106, e.g., using hook-and-loop fasteners, or another type of fastener.

Before the superior compliant layer 3120 is deposited, or otherwise affixed to the superior bearing surface 3106, the superior bearing surface 3106 can be modified to promote adhesion of the superior compliant layer 3120 to the superior bearing surface 3106. For example, the superior bearing surface 3106 can be roughened to promote adhesion of the superior compliant layer 3120. For example, the roughening process can include acid etching; knurling; application of a bead coating, e.g., cobalt chrome beads; application of a roughening spray; e.g., titanium plasma spray (TPS); laser blasting; or any other similar process or method.

In a particular embodiment, after installation, the superior compliant layer 3120 can be in direct contact with vertebral bone, e.g., cortical bone and cancellous bone. In a particular embodiment, the superior compliant layer 3120 can be an extended use biocompatible material. For example, the extended use biocompatible materials can include synthetic polymers, natural polymers, bioactive ceramics, compression molded carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the superior compliant layer 3120 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 30 through FIG. 33 indicate that the superior component 3100 can include a superior keel 3148 that extends from superior bearing surface 3106. During installation, described below, the superior keel 3148 can at least partially engage a keel groove that can be established within a cortical rim of a vertebra. Further, the superior keel 3148 can be coated with a bone-growth promoting substance, e.g., a hydroxyapatite coating formed of calcium phosphate. In a particular embodiment, the superior keel 3148 does not include proteins, e.g., bone morphogenetic protein (BMP). Additionally, the superior keel 3148 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the superior component 3100, depicted in FIG. 33, can be generally rectangular in shape. For example, the superior component 3100 can have a substantially straight posterior side 3150. A first substantially straight lateral side 3152 and a second substantially straight lateral side 3154 can extend substantially perpendicularly from the posterior side 3150 to an anterior side 3156. In a particular embodiment, the anterior side 3156 can curve outward such that the superior component 3100 is wider through the middle than along the lateral sides 3152, 3154. Further, in a particular embodiment, the lateral sides 3152, 3154 are substantially the same length.

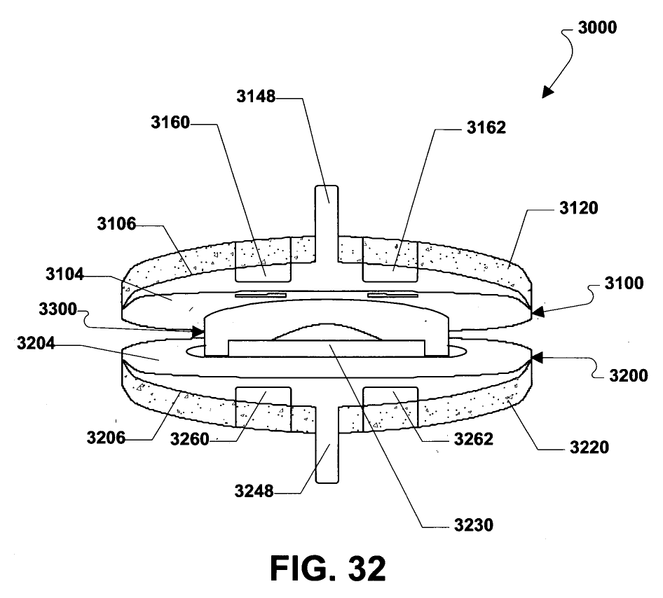

FIG. 32 and FIG. 33 show that the superior component 3100 can include a first implant inserter engagement hole 3160 and a second implant inserter engagement hole 3162. In a particular embodiment, the implant inserter engagement holes 3160, 3162 are configured to receive a correspondingly shaped arm that extends from an implant inserter (not shown) that can be used to facilitate the proper installation of an intervertebral prosthetic disc, e.g., the intervertebral prosthetic disc 3000 shown in FIG. 30 through FIG. 34.

In a particular embodiment, the inferior component 3200 includes an inferior support plate 3202 that has an inferior articular surface 3204 and an inferior bearing surface 3206. In a particular embodiment, the inferior articular surface 3204 can be substantially flat and the inferior bearing surface 3206 can be generally curved. In an alternative embodiment, at least a portion of the inferior articular surface 3204 can be generally curved and the inferior bearing surface 3206 can be substantially flat.

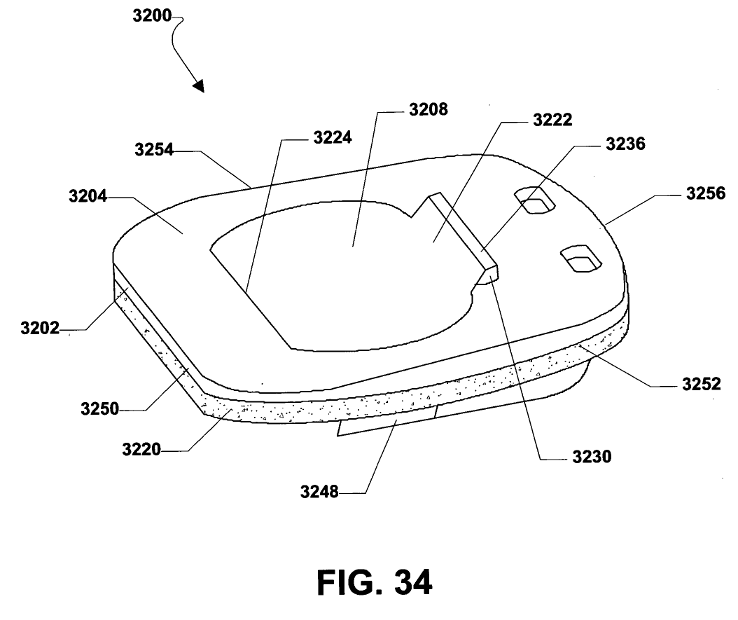

As illustrated in FIG. 34, an inferior depression 3208 is established within the inferior articular surface 3204 of the inferior support plate 3202. In a particular embodiment, the inferior depression 3208 has an arcuate shape. For example, the inferior depression 3208 can have a hemispherical shape, an elliptical shape, a cylindrical shape, or any combination thereof.

As further illustrated, the inferior component 3200 includes an inferior compliant layer 3220 that can be affixed to, attached to, or otherwise deposited on, the inferior bearing surface 3206. As shown, the inferior compliant layer 3220 can be substantially convex. Further, the inferior compliant layer 3220 can have a thickness that is substantially uniform. Alternatively, the inferior compliant layer 3220 can have a thickness that varies throughout the inferior compliant layer 3220.

The inferior compliant layer 3220 can be chemically bonded to the inferior bearing surface 3206, e.g., using an adhesive or another chemical bonding agent. Further, the inferior compliant layer 3220 can be mechanically anchored to the inferior bearing surface 3206, e.g., using hook-and-loop fasteners, or another type of fastener.

Before the inferior compliant layer 3220 is deposited, or otherwise affixed to the inferior bearing surface 3206, the inferior bearing surface 3206 can be modified to promote adhesion of the inferior compliant layer 3220 to the inferior bearing surface 3206. For example, the inferior bearing surface 3206 can be roughened to promote adhesion of the inferior compliant layer 3220. For example, the roughening process can include acid etching; knurling; application of a bead coating, e.g., cobalt chrome beads; application of a roughening spray; e.g., titanium plasma spray (TPS); laser blasting; or any other similar process or method.

In a particular embodiment, after installation, the inferior compliant layer 3220 can be in direct contact with vertebral bone, e.g., cortical bone and cancellous bone. In a particular embodiment, the inferior compliant layer 3220 can be an extended use biocompatible material. For example, the extended use biocompatible materials can include synthetic polymers, natural polymers, bioactive ceramics, compression molded carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the inferior compliant layer 3220 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

As further shown in FIG. 34, the inferior depression 3208 includes an anterior rim 3222 and a poster rim 3224. Further, an inferior nucleus containment rail 3230 extends from the inferior articular surface 3204 adjacent to the anterior rim 3222 of the inferior depression 3208. As shown in FIG. 34, the inferior nucleus containment rail 3230 is an extension of the surface of the inferior depression 3208. In a particular embodiment, as shown in FIG. 30, the inferior nucleus containment rail 3230 extends into a gap 3234 that can be established between the superior component 3100 and the inferior component 3200 posterior to the nucleus 3300. Further, the inferior nucleus containment rail 3230 can include a slanted upper surface 3236. In a particular embodiment, the slanted upper surface 3236 of the inferior nucleus containment rail 3230 can prevent the inferior nucleus containment rail 3230 from interfering with the motion of the superior component 3100 with respect to the inferior component 3200.

In lieu of, or in addition to, the inferior nucleus containment rail 3230, a superior nucleus containment rail (not shown) can extend from the superior articular surface 3104 of the superior component 3100. In a particular embodiment, the superior nucleus containment rail (not shown) can be configured substantially identical to the inferior nucleus containment rail 3230. In various alternative embodiments (not shown), each or both of the superior component 3100 and the inferior component 3200 can include multiple nucleus containment rails extending from the respective articular surfaces 3104, 3204. The containment rails can be staggered or provided in other configurations based on the perceived need to prevent nucleus migration in a given direction.

FIG. 30 through FIG. 32 and FIG. 34 indicate that the inferior component 3200 can include an inferior keel 3248 that extends from inferior bearing surface 3206. During installation, described below, the inferior keel 3248 can at least partially engage a keel groove that can be established within a cortical rim of a vertebra. Further, the inferior keel 3248 can be coated with a bone-growth promoting substance, e.g., a hydroxyapatite coating formed of calcium phosphate. In a particular embodiment, the inferior keel 3248 does not include proteins, e.g., bone morphogenetic protein (BMP). Additionally, the inferior keel 3248 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the inferior component 3200, shown in FIG. 34, can be shaped to match the shape of the superior component 3100, shown in FIG. 33. Further, the inferior component 3200 can be generally rectangular in shape. For example, the inferior component 3200 can have a substantially straight posterior side 3250. A first substantially straight lateral side 3252 and a second substantially straight lateral side 3254 can extend substantially perpendicularly from the posterior side 3250 to an anterior side 3256. In a particular embodiment, the anterior side 3256 can curve outward such that the inferior component 3200 is wider through the middle than along the lateral sides 3252, 3254. Further, in a particular embodiment, the lateral sides 3252, 3254 are substantially the same length.

FIG. 32 and FIG. 34 show that the inferior component 3200 can include a first implant inserter engagement hole 3260 and a second implant inserter engagement hole 3262. In a particular embodiment, the implant inserter engagement holes 3260, 3262 are configured to receive a correspondingly shaped arm that extends from an implant inserter (not shown) that can be used to facilitate the proper installation of an intervertebral prosthetic disc, e.g., the intervertebral prosthetic disc 3000 shown in FIG. 30 through FIG. 34.

FIG. 32 shows that the nucleus 3300 can include a superior bearing surface 3302 and an inferior bearing surface 3304. In a particular embodiment, the superior bearing surface 3302 and the inferior bearing surface 3304 can each have an arcuate shape. For example, the superior bearing surface 3302 of the nucleus 3300 and the inferior bearing surface 3304 of the nucleus 3300 can have a hemispherical shape, an elliptical shape, a cylindrical shape, or any combination thereof. Further, in a particular embodiment, the superior bearing surface 3302 can be curved to match the superior depression 3108 of the superior component 3100. Also, in a particular embodiment, the inferior bearing surface 3304 of the nucleus can be curved to match the inferior depression 3208 of the inferior component 3200.

As shown in FIG. 30, the superior bearing surface 3302 of the nucleus 3300 can engage the superior depression 3108 and allow the superior component 3100 to move relative to the nucleus 3300. Also, the inferior bearing surface 3304 of the nucleus 3300 can engage the inferior depression 3208 and allow the inferior component 3200 to move relative to the nucleus 3300. Accordingly, the nucleus 3300 can engage the superior component 3100 and the inferior component 3200 and the nucleus 3300 can allow the superior component 3100 to rotate with respect to the inferior component 3200.

In a particular embodiment, the inferior nucleus containment rail 3230 on the inferior component 3200 can prevent the nucleus 3300 from migrating, or moving, with respect to the superior component 3100, the inferior component 3200, or a combination thereof. In other words, the inferior nucleus containment rail 3230 can prevent the nucleus 3300 from moving out of the superior depression 3108, the inferior depression 3208, or a combination thereof.

Further, the inferior nucleus containment rail 3230 can prevent the nucleus 3300 from being expelled from the intervertebral prosthetic device 3000. In other words, the inferior nucleus containment rail 3230 on the inferior component 3200 can prevent the nucleus 3300 from being completely ejected from the intervertebral prosthetic device 3000 while the superior component 3100 and the inferior component 3200 move with respect to each other.

In a particular embodiment, the overall height of the intervertebral prosthetic device 3000 can be in a range from fourteen millimeters to forty-six millimeters (14-46 mm). Further, the installed height of the intervertebral prosthetic device 3000 can be in a range from eight millimeters to sixteen millimeters (8-16 mm). In a particular embodiment, the installed height can be substantially equivalent to the distance between an inferior vertebra and a superior vertebra when the intervertebral prosthetic device 3000 is installed there between.

In a particular embodiment, the length of the intervertebral prosthetic device 3000, e.g., along a longitudinal axis, can be in a range from thirty millimeters to forty millimeters (30-40 mm). Additionally, the width of the intervertebral prosthetic device 3000, e.g., along a lateral axis, can be in a range from twenty-five millimeters to forty millimeters (25-40 mm). Moreover, in a particular embodiment, each keel 3148, 3248 can have a height in a range from three millimeters to fifteen millimeters (3-15 mm).

Description of a Fifth Embodiment of an Intervertebral Prosthetic Disc

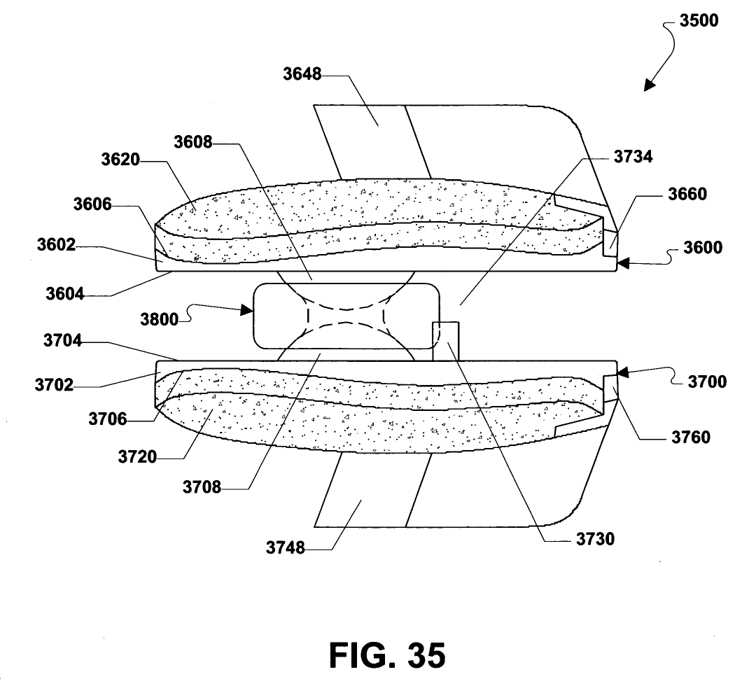

Referring to FIGS. 35 through 39, a fifth embodiment of an intervertebral prosthetic disc is shown and is generally designated 3500. As illustrated, the intervertebral prosthetic disc 3500 includes a superior component 3600, an inferior component 3700, and a nucleus 3800 disposed, or otherwise installed, there between. In a particular embodiment, the components 3600, 3700 and the nucleus 3800 can be made from one or more extended use biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 3600, 3700 can be made from any other substantially rigid biocompatible materials.

In a particular embodiment, the superior component 3600 includes a superior support plate 3602 that has a superior articular surface 3604 and a superior bearing surface 3606. In a particular embodiment, the superior articular surface 3604 can be substantially flat and the superior bearing surface 3606 can be generally curved. In an alternative embodiment, at least a portion of the superior articular surface 3604 can be generally curved and the superior bearing surface 3606 can be substantially flat.

As illustrated in FIG. 35 through FIG. 38, a superior projection 3608 extends from the superior articular surface 3604 of the superior support plate 3602. In a particular embodiment, the superior projection 3608 has an arcuate shape. For example, the superior depression 3608 can have a hemispherical shape, an elliptical shape, a cylindrical shape, or any combination thereof.

As further illustrated, the superior component 3600 includes a superior compliant layer 3620 that can be affixed to, attached to, or otherwise deposited on, the superior bearing surface 3606. As shown, the superior compliant layer 3620 can be substantially convex. Further, the superior compliant layer 3620 can have a thickness that is substantially uniform. Alternatively, the superior compliant layer 3620 can have a thickness that varies throughout the superior compliant layer 3620.

The superior compliant layer 3620 can be chemically bonded to the superior bearing surface 3606, e.g., using an adhesive or another chemical bonding agent. Further, the superior compliant layer 3620 can be mechanically anchored to the superior bearing surface 3606, e.g., using hook-and-loop fasteners, or another type of fastener.

Before the superior compliant layer 3620 is deposited, or otherwise affixed to the superior bearing surface 3606, the superior bearing surface 3606 can be modified to promote adhesion of the superior compliant layer 3620 to the superior bearing surface 3606. For example, the superior bearing surface 3606 can be roughened to promote adhesion of the superior compliant layer 3620. For example, the roughening process can include acid etching; knurling; application of a bead coating, e.g., cobalt chrome beads; application of a roughening spray; e.g., titanium plasma spray (TPS); laser blasting; or any other similar process or method.

In a particular embodiment, after installation, the superior compliant layer 3620 can be in direct contact with vertebral bone, e.g., cortical bone and cancellous bone. In a particular embodiment, the superior compliant layer 3620 can be an extended use biocompatible material. For example, the extended use biocompatible materials can include synthetic polymers, natural polymers, bioactive ceramics, compression molded carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the superior compliant layer 3620 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 35 through FIG. 38 indicate that the superior component 3600 can include a superior keel 3648 that extends from superior bearing surface 3606. During installation, described below, the superior keel 3648 can at least partially engage a keel groove that can be established within a cortical rim of a vertebra. Further, the superior keel 3648 can be coated with a bone-growth promoting substance, e.g., a hydroxyapatite coating formed of calcium phosphate. In a particular embodiment, the superior keel 3648 does not include proteins, e.g., bone morphogenetic protein (BMP). Additionally, the superior keel 3648 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

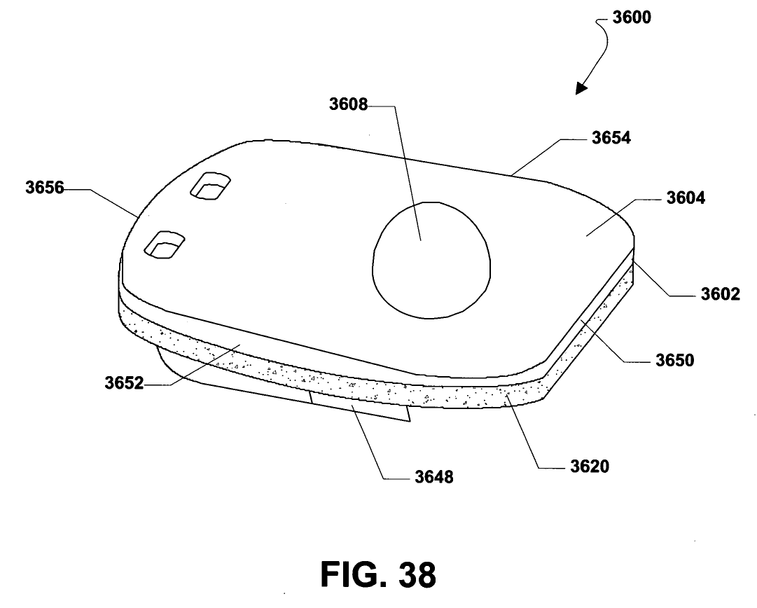

In a particular embodiment, the superior component 3600, depicted in FIG. 38, can be generally rectangular in shape. For example, the superior component 3600 can have a substantially straight posterior side 3650. A first substantially straight lateral side 3652 and a second substantially straight lateral side 3654 can extend substantially perpendicularly from the posterior side 3650 to an anterior side 3656. In a particular embodiment, the anterior side 3656 can curve outward such that the superior component 3600 is wider through the middle than along the lateral sides 3652, 3654. Further, in a particular embodiment, the lateral sides 3652, 3654 are substantially the same length.

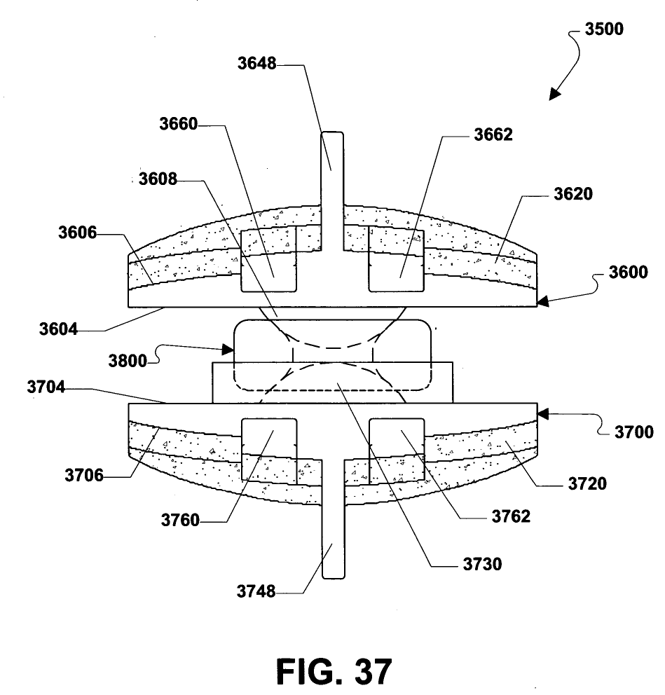

FIG. 37 and FIG. 38 show that the superior component 3600 can include a first implant inserter engagement hole 3660 and a second implant inserter engagement hole 3662. In a particular embodiment, the implant inserter engagement holes 3660, 3662 are configured to receive a correspondingly shaped arm that extends from an implant inserter (not shown) that can be used to facilitate the proper installation of an intervertebral prosthetic disc, e.g., the intervertebral prosthetic disc 3500 shown in FIG. 35 through FIG. 39.

In a particular embodiment, the inferior component 3700 includes an inferior support plate 3702 that has an inferior articular surface 3704 and an inferior bearing surface 3706. In a particular embodiment, the inferior articular surface 3704 can be substantially flat and the inferior bearing surface 3706 can be generally curved. In an alternative embodiment, at least a portion of the inferior articular surface 3704 can be generally curved and the inferior bearing surface 3706 can be substantially flat.

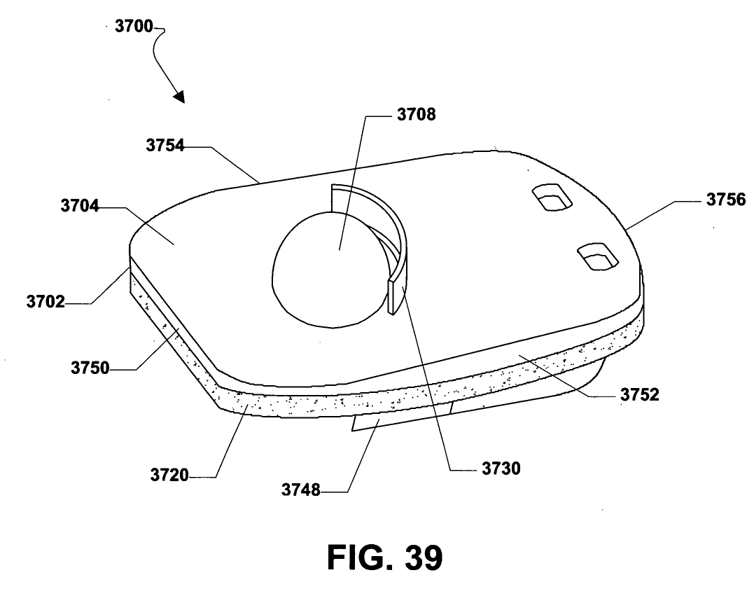

As illustrated in FIG. 39, an inferior projection 3708 can extend from the inferior articular surface 3704 of the inferior support plate 3702. In a particular embodiment, the inferior projection 3708 has an arcuate shape. For example, the inferior projection 3708 can have a hemispherical shape, an elliptical shape, a cylindrical shape, or any combination thereof.

As further illustrated, the inferior component 3700 includes an inferior compliant layer 3720 that can be affixed to, attached to, or otherwise deposited on, the inferior bearing surface 3706. As shown, the inferior compliant layer 3720 can be substantially convex. Further, the inferior compliant layer 3720 can have a thickness that is substantially uniform. Alternatively, the inferior compliant layer 3720 can have a thickness that varies throughout the inferior compliant layer 3720.

The inferior compliant layer 3720 can be chemically bonded to the inferior bearing surface 3706, e.g., using an adhesive or another chemical bonding agent. Further, the inferior compliant layer 3720 can be mechanically anchored to the inferior bearing surface 3706, e.g., using hook-and-loop fasteners, or another type of fastener.

Before the inferior compliant layer 3720 is deposited, or otherwise affixed to the inferior bearing surface 3706, the inferior bearing surface 3706 can be modified to promote adhesion of the inferior compliant layer 3720 to the inferior bearing surface 3706. For example, the inferior bearing surface 3706 can be roughened to promote adhesion of the inferior compliant layer 3720. For example, the roughening process can include acid etching; knurling; application of a bead coating, e.g., cobalt chrome beads; application of a roughening spray; e.g., titanium plasma spray (TPS); laser blasting; or any other similar process or method.

In a particular embodiment, after installation, the inferior compliant layer 3720 can be in direct contact with vertebral bone, e.g., cortical bone and cancellous bone. In a particular embodiment, the inferior compliant layer 3720 can be an extended use biocompatible material. For example, the extended use biocompatible materials can include synthetic polymers, natural polymers, bioactive ceramics, compression molded carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the inferior compliant layer 3720 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

As further shown, an inferior nucleus containment rail 3730 can extend from the inferior articular surface 3704 adjacent to the inferior projection 3708. As shown in FIG. 39, the inferior nucleus containment rail 3730 is a curved wall that extends from the inferior articular surface 3704. In a particular embodiment, the inferior nucleus containment rail 3730 can be curved to match the shape, or curvature, of the inferior projection 3708. Alternatively, the inferior nucleus containment rail 3730 can be curved to match the shape, or curvature, of the nucleus 3800. In a particular embodiment, the inferior nucleus containment rail 3730 extends into a gap 3734 that can be established between the superior component 3600 and the inferior component 3700 posterior to the nucleus 3800.

In lieu of, or in addition to, the inferior nucleus containment rail 3730, a superior nucleus containment rail (not shown) can extend from the superior articular surface 3604 of the superior component 3600. In a particular embodiment, the superior nucleus containment rail (not shown) can be configured substantially identical to the inferior nucleus containment rail 3730. In various alternative embodiments (not shown), each or both of the superior component 3600 and the inferior component 3700 can include multiple nucleus containment rails extending from the respective articular surfaces 3604, 3704. The containment rails can be staggered or provided in other configurations based on the perceived need to prevent nucleus migration in a given direction.

FIG. 35 through FIG. 37 and FIG. 39 indicate that the inferior component 3700 can include an inferior keel 3748 that extends from inferior bearing surface 3706. During installation, described below, the inferior keel 3748 can at least partially engage a keel groove that can be established within a cortical rim of a vertebra. Further, the inferior keel 3748 can be coated with a bone-growth promoting substance, e.g., a hydroxyapatite coating formed of calcium phosphate. In a particular embodiment, the inferior keel 3748 does not include proteins, e.g., bone morphogenetic protein (BMP). Additionally, the inferior keel 3748 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the inferior component 3700, shown in FIG. 39, can be shaped to match the shape of the superior component 3600, shown in FIG. 38. Further, the inferior component 3700 can be generally rectangular in shape. For example, the inferior component 3700 can have a substantially straight posterior side 3750. A first substantially straight lateral side 3752 and a second substantially straight lateral side 3754 can extend substantially perpendicularly from the posterior side 3750 to an anterior side 3756. In a particular embodiment, the anterior side 3756 can curve outward such that the inferior component 3700 is wider through the middle than along the lateral sides 3752, 3754. Further, in a particular embodiment, the lateral sides 3752, 3754 are substantially the same length.

FIG. 37 and FIG. 39 show that the inferior component 3700 can include a first implant inserter engagement hole 3760 and a second implant inserter engagement hole 3762. In a particular embodiment, the implant inserter engagement holes 3760, 3762 are configured to receive a correspondingly shaped arm that extends from an implant inserter (not shown) that can be used to facilitate the proper installation of an intervertebral prosthetic disc, e.g., the intervertebral prosthetic disc 3500 shown in FIG. 35 through FIG. 39.

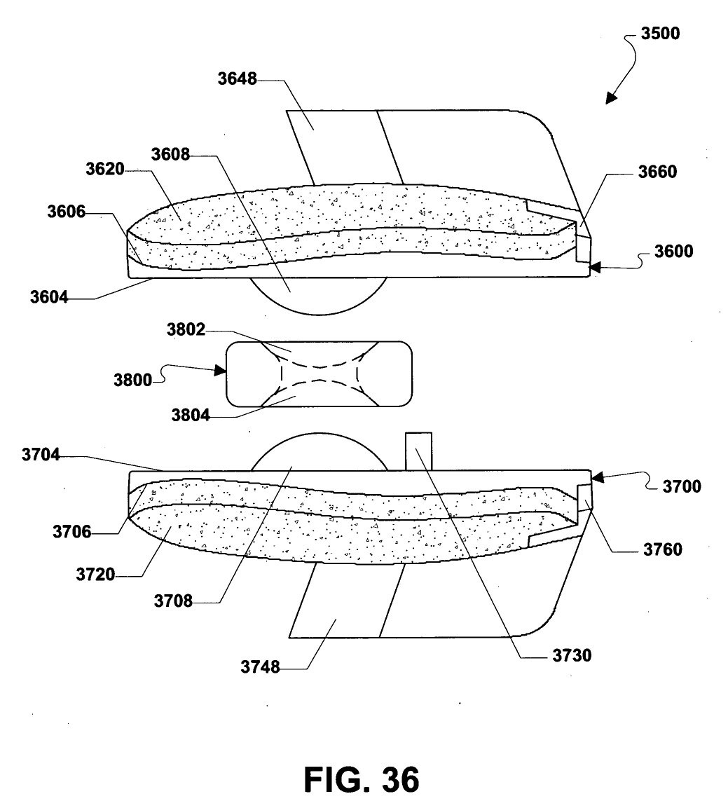

FIG. 36 shows that the nucleus 3800 can include a superior depression 3802 and an inferior depression 3804. In a particular embodiment, the superior depression 3802 and the inferior depression 3804 can each have an arcuate shape. For example, the superior depression 3802 of the nucleus 3800 and the inferior depression 3804 of the nucleus 3800 can have a hemispherical shape, an elliptical shape, a cylindrical shape, or any combination thereof. Further, in a particular embodiment, the superior depression 3802 can be curved to match the superior projection 3608 of the superior component 3600. Also, in a particular embodiment, the inferior depression 3804 of the nucleus 3800 can be curved to match the inferior projection 3708 of the inferior component 3700.

As shown in FIG. 35, the superior depression 3802 of the nucleus 3800 can engage the superior projection 3608 and allow the superior component 3600 to move relative to the nucleus 3800. Also, the inferior depression 3804 of the nucleus 3800 can engage the inferior projection 3708 and allow the inferior component 3700 to move relative to the nucleus 3800. Accordingly, the nucleus 3800 can engage the superior component 3600 and the inferior component 3700, and the nucleus 3800 can allow the superior component 3600 to rotate with respect to the inferior component 3700.

In a particular embodiment, the inferior nucleus containment rail 3730 on the inferior component 3700 can prevent the nucleus 3800 from migrating, or moving, with respect to the superior component 3600 and the inferior component 3700. In other words, the inferior nucleus containment rail 3730 can prevent the nucleus 3800 from moving off of the superior projection 3608, the inferior projection 3708, or a combination thereof.

Further, the inferior nucleus containment rail 3730 can prevent the nucleus 3800 from being expelled from the intervertebral prosthetic device 3500. In other words, the inferior nucleus containment rail 3730 on the inferior component 3700 can prevent the nucleus 3800 from being completely ejected from the intervertebral prosthetic device 3500 while the superior component 3600 and the inferior component 3700 move with respect to each other.

In a particular embodiment, the overall height of the intervertebral prosthetic device 3500 can be in a range from fourteen millimeters to forty-six millimeters (14-46 mm). Further, the installed height of the intervertebral prosthetic device 3500 can be in a range from eight millimeters to sixteen millimeters (8-16 mm). In a particular embodiment, the installed height can be substantially equivalent to the distance between an inferior vertebra and a superior vertebra when the intervertebral prosthetic device 3500 is installed there between.

In a particular embodiment, the length of the intervertebral prosthetic device 3500, e.g., along a longitudinal axis, can be in a range from thirty millimeters to forty millimeters (30-40 mm). Additionally, the width of the intervertebral prosthetic device 3500, e.g., along a lateral axis, can be in a range from twenty-five millimeters to forty millimeters (25-40 mm). Moreover, in a particular embodiment, each keel 3648, 3748 can have a height in a range from three millimeters to fifteen millimeters (3-15 mm).

### CONCLUSION

With the configuration of structure described above, the intervertebral prosthetic disc according to one or more of the embodiments provides a device that may be implanted to replace a natural intervertebral disc that is diseased, degenerated, or otherwise damaged. The intervertebral prosthetic disc can be disposed within an intervertebral space between an inferior vertebra and a superior vertebra. Further, after a patient fully recovers from a surgery to implant the intervertebral prosthetic disc, the intervertebral prosthetic disc can provide relative motion between the inferior vertebra and the superior vertebra that closely replicates the motion provided by a natural intervertebral disc. Accordingly, the intervertebral prosthetic disc provides an alternative to a fusion device that can be implanted within the intervertebral space between the inferior vertebra and the superior vertebra to fuse the inferior vertebra and the superior vertebra and prevent relative motion there between.

The compliant layers of the intervertebral prosthetic disc can allow the intervertebral prosthetic disc to conform to the shapes of the vertebrae between which the intervertebral prosthetic disc is implanted. Full conformance can increase the surface area for osteointegration, which, in turn, can prevent, or substantially minimize, the chance of the intervertebral prosthetic disc becoming loose during the lifetime of the intervertebral prosthetic disc.

The above-disclosed subject matter is to be considered illustrative, and not restrictive, and the appended claims are intended to cover all such modifications, enhancements, and other embodiments that fall within the true spirit and scope of the present invention. For example, it is noted that the components in the exemplary embodiments described herein are referred to as “superior” and “inferior” for illustrative purposes only and that one or more of the features described as part of or attached to a respective half may be provided as part of or attached to the other half in addition or in the alternative. Thus, to the maximum extent allowed by law, the scope of the present invention is to be determined by the broadest permissible interpretation of the following claims and their equivalents, and shall not be restricted or limited by the foregoing detailed description.

1. An intervertebral prosthetic disc to be installed within an intervertebral space between a first vertebra and a second vertebra, the intervertebral prosthetic disc comprising:
a first component comprising a first compliant layer configured to engage the first vertebra and at least partially conform to a shape of the first vertebra; and
a second component configured to engage the second vertebra.

2. The intervertebral prosthetic disc of claim 1, wherein the first compliant layer is at least partially convex.

3. The intervertebral prosthetic disc of claim 1, wherein the first compliant layer has a substantially uniform thickness.

4. The intervertebral prosthetic disc of claim 1, wherein the first compliant layer has a variable thickness.

5. The intervertebral prosthetic disc of claim 1, wherein the first compliant layer comprises an extended use biocompatible material.

6. The intervertebral prosthetic disc of claim 5, wherein the extended use biocompatible material is a synthetic polymer, a natural polymer, a bioactive ceramic, compression molded carbon nanofibers, or a combination thereof.

7. The intervertebral prosthetic disc of claim 6, wherein the synthetic polymer is a polyurethane material, a polyolefin material, a polyether material, a polyester material, a polycarbonate material, a silicone material, a hydrogel material, or a combination thereof.

8. The intervertebral prosthetic disc of claim 7, wherein the polyolefin material is polypropylene, polyethylene, halogenated polyolefin, flouropolyolefin, or a combination thereof.

9. The intervertebral prosthetic disc of claim 7, wherein the polyether material is polyetherketone (PEK), polyetheretherketone (PEEK), polyetherketoneketone (PEKK), polyaryletherketone (PAEK), or a combination thereof.

10. The intervertebral prosthetic disc of claim 7, wherein the polyester material is polylactide.

11. The intervertebral prosthetic disc of claim 7, wherein the polycarbonate material is tyrosine polycarbonate.

12. The intervertebral prosthetic disc of claim 6, wherein the natural polymer is collagen, gelatin, fibrin, keratin, chitosan, chitin, hyaluronic acid, albumin, silk, elastin, or a combination thereof.

13. The intervertebral prosthetic disc of claim 6, wherein the bioactive ceramic is hydroxyapatite (HA), hydroxyapatite tricalcium phosphate (HATCP), calcium phosphate, calcium sulfate, or a combination thereof.

14. The intervertebral prosthetic disc of claim 1, wherein the superior compliant layer, the inferior compliant layer, or a combination thereof includes a biological factor to promote bone growth.

15. The intervertebral prosthetic disc of claim 14, wherein the biological factor is a bone morphogenetic protein (BMP), a cartilage-derived morphogenetic protein (CDMP), a platelet derived growth factor (PDGF), an insulin-like growth factor (IGF), a LIM mineralization protein, a fibroblast growth factor (FGF), an osteoblast growth factor, stem cells, or a combination thereof.

16. The intervertebral prosthetic disc of claim 15, wherein the stem cells include bone marrow derived stem cells, lipo derived stem cells, or a combination thereof.

17. The intervertebral prosthetic disc of claim 1, further comprising a first embedded structure at least partially disposed within the first compliant layer.

18. The intervertebral prosthetic disc of claim 17, wherein the first embedded structure is substantially non-resorbable.

19. The intervertebral prosthetic disc of claim 18, wherein the first compliant layer is substantially resorbable.

20. The intervertebral prosthetic disc of claim 17, wherein the first embedded structure comprises a fabric mesh, a metallic mesh, a polyetheretherketone mesh, a three-dimensional polyester structure, or a combination thereof.

21. The intervertebral prosthetic disc of claim 1, wherein the first component further comprises a first keel at least partially covered by the first compliant layer.

22. The intervertebral prosthetic disc of claim 21, wherein the first component further comprises a first tooth at least partially covered by the first compliant layer.

23. The intervertebral prosthetic disc of claim 22, wherein the first keel, the first tooth, or a combination thereof is configured to at least partially extend through the first compliant layer and engage the first vertebra.

24. The intervertebral prosthetic disc of claim 1, wherein the second component comprises a second compliant layer configured to engage the second vertebra and at least partially conform to a shape of the second vertebra.

25. The intervertebral prosthetic disc of claim 24, wherein the first compliant layer is at least partially convex and the second compliant layer is at least partially convex.

26. The intervertebral prosthetic disc of claim 24, wherein the first compliant layer has a substantially uniform thickness and the second compliant layer has a substantially uniform thickness.

27. The intervertebral prosthetic disc of claim 24, wherein the first compliant layer has a variable thickness and the second compliant layer has a variable thickness.

28. The intervertebral prosthetic disc of claim 24, further comprising a second embedded structure at least partially disposed within the second compliant layer.

29. The intervertebral prosthetic disc of claim 24, further comprising a second keel at least partially covered by the second compliant layer.

30. The intervertebral prosthetic disc of claim 29, further comprising a second at least partially covered by the second compliant layer.

31. The intervertebral prosthetic disc of claim 30, wherein the second keel, the second tooth, or a combination thereof is configured to at least partially extend through the second compliant layer and engage the second vertebra.

32. An intervertebral prosthetic disc to be installed within an intervertebral space between an inferior vertebra and a superior vertebra, the intervertebral prosthetic disc comprising:
an inferior support plate having an inferior bearing surface;
an inferior compliant layer disposed on the inferior bearing surface;
an inferior embedded layer disposed within the inferior bearing surface;
a superior support plate having a superior bearing surface;
a superior compliant layer disposed on the superior bearing surface; and
a superior embedded layer disposed within the superior bearing surface.

33.-45. (canceled)

46. An intervertebral prosthetic disc to be installed within an intervertebral space between an inferior vertebra and a superior vertebra, the intervertebral prosthetic disc comprising:
a superior component, the superior component comprising:
a superior support plate having a superior bearing surface; and
a superior compliant layer disposed on the superior bearing surface;
an inferior component, the inferior component comprising:
an inferior support plate having an inferior bearing surface; and
an inferior compliant layer disposed on the inferior bearing surface; and
a nucleus disposed between the superior component and the inferior component, wherein the nucleus is configured to allow relative motion between the superior component and the inferior component.

47.-51. (canceled)
